(19) United States
(12) Patent Application Publication
(10) Pub. No.: US 2008/0269789 A1
(43) Pub. Date: Oct. 30, 2008

(54) Implantable device with miniature rotating portion for the treatment of atherosclerosis, especially vulnerable plaques

(76) Inventor: Uri Eli, Kiryat Ono (IL)

Correspondence Address: Dr. D. Graeser Ltd.;c/o The Discovery Dispatch, 9003 Florin Way, Upper Marlboro, MD, 20772, US

(21) Appl. No.: 11/976,435

(22) Filed: Oct. 24, 2007

Related U.S. Application Data
(60) Provisional application No. 60/924,041, filed on Apr. 27, 2007.

(51) Int. Cl. A61B 17/22

(52) U.S. Cl. 606/159

(57) ABSTRACT

A miniature rotating portion, anchored to and used within the human body. Optionally and preferably, the device may be used for one or more of active filtration and removal of plaques in the blood vessel, acting as an active plaque disassembler and lysis accelerator, acting as a localized sensor and/or acting as a hydrokinetic power generator.

This Application claims priority from U.S. Provisional Application No. 60/924,041, filed on Apr. 27, 2007, which is hereby incorporated by reference as if fully set forth herein.

### FIELD OF THE INVENTION

The present invention relates to implantable devices with at least one miniature rotating portion, and in particular to such devices for use in the human body to treat atherosclerosis in general and vulnerable plaque in particular within the cardiovascular system.

### BACKGROUND OF THE INVENTION

Atherosclerosis is the name of the process in which deposits of fatty substances, cholesterol, cellular waste products, calcium and other substances build up in the inner lining of an artery. This build up is called plaque. Some hardening of arteries often occurs when people grow older. Plaques can grow large enough to significantly reduce the blood's flow through an artery, although most of the damage occurs when they become fragile and rupture.

Atherosclerosis, including coronary artery disease, affects nearly 14 million people in the United States alone and is the leading cause of death in the United States. It is the cause of acute myocardial infarction (MI) and strokes. Some plaques are stable plaque with a thick calcified cap and a smaller fatty core, while other plaques are unstable plaques with a thin calcified cap covering a larger fatty core. Recently, this unstable and soft plaque has been called “vulnerable plaque” because of its tendency to burst or rupture. Unstable plaque is more likely to rupture, which can trigger a heart attack or a stroke. In the past, it was believed that most heart disease was the result of a progressive increase of hard plaque in the coronary arteries, which may lead to the complete occlusion of an artery. This narrowing (stenosis) process reduces blood flow, which triggers the formation of a blood clot. The clot may reduce or even eliminate the flow of oxygen rich blood to heart muscles, causing ischemia and ultimately a heart attack. Alternatively, the clot may break off and travel to the brain resulting in a thrombotic stroke.

Once a clogged vessel has been identified, it is commonly treated by implanting a stent. Stents are particularly useful in the treatment of atherosclerotic stenosis in arteries and blood vessels. An intravascular interventional device such as a stent is particularly useful in the treatment and repair of blood vessels after a stenosis has been treated by percutaneous transluminal coronary angioplasty (PTCA), percutaneous transluminal angioplasty (PTA), or removed by atherectomy or other means, to help improve the results of the procedure and reduce the possibility of restenosis. Stents also can be used to provide primary compression to a stenosis in cases in which no initial PTCA or PTA procedure is performed.

However, it is now believed that rupture of a nonstenotic, yet vulnerable atherosclerotic plaque, frequently leads to an acute coronary syndrome and strokes. It is believed that physical disruption of such a plaque allows circulating blood coagulation factors to interact with the highly thrombogenic material in the plaque's lipid core, thereby instigating the formation of a potentially occluding and fatal thrombus.

Mechanical stress and composition of plaques play an important role in plaque disruption. Mechanical forces can easily disrupt this plaque, even the mere vibration of the heart as it beats. The plaques are classified as either yellow or white using coronary angioscopy. Yellow plaques with an increased distensibility and a compensatory enlargement may be mechanically and structurally weak. As a result, mechanical “fatigue,” caused by repetitive stretching, may lead to plaque disruption. Plaques with a high distensibility and a compensatory enlargement may be vulnerable.

The development of vulnerable plaques is not limited to the localized lesions but is a pan-coronary process. In patients with myocardial infarction, all three major coronary arteries are typically diseased and have multiple yellow plaques that are undisrupted.

One of the most important issues of vulnerable plaque is the fact that vulnerable plaques do not bulge inward. Instead, as plaque grows, it often protrudes outward, into the wall of the artery, rather than into the channel-lumen where blood flows. On an angiogram, the vessel may appear to be normal. But when dissected after death, the arteries' walls are thick with plaque that could not yet be seen by angiogram.

Although much work is focused on the field of vulnerable plaque, it is hindered by lack of diagnostic tools that can visualize vulnerable plaque in patients. Current modalities like angiography grossly underestimate the presence of arterial disease. Intra-vascular ultrasound (IVUS), which is currently used to place stents and find occlusive plaques, likewise doesn't have the resolution or penetration necessary to examine details of the vascular wall.

At present, there are several methods that allow health care givers to view vulnerable plaques. Several invasive and non-invasive imaging techniques are available to assess atherosclerotic disease vessels for example including Magnetic Resonance Imaging (MRI), thermal sensors that measure the temperature of the arterial wall on the premise that the inflammatory process at the root of the problem generates heat, elasticity sensors, better kinds of intra-vascular ultrasound, optical coherence tomography (OCT), contrast agents, infrared and near-infrared light spectroscopy, and blood tests that may identify proteins resulting from inflammation of the arteries, or the like.

### SUMMARY OF THE INVENTION

There is an unmet need for, and it would be highly useful to have, a device for detecting vulnerable plaques. There is also an unmet need for, and it would be highly useful to have, a device for decreasing a likelihood of rupture in a way that can cause coronary occlusion, atherosclerosis or arteriosclerosis.

There is also an unmet need for, and it would be highly useful to have, a device that is able to treat vulnerable plaque and protect a blood vessel and body tissues against damage caused by plaque rupture.

The present invention overcomes these disadvantages of the background art by harnessing a small portion of the freely available hydrokinetic energy inherent in the human body blood flow and converting it into mechanical, electrical, electrostatic, optical, magnetic, or RF power or other forms of useable and preferable transmittable energy.

The different optional embodiments of the present invention provide for a device and method for protecting a blood vessel, and hence bodily tissues, from damage caused by particulate such as ruptured plaque, and treating the vulnerable plaque in order to stabilize it.

Blood vessel protection from plaque rupture may be gained by not only by “isolating” the vulnerable plaque from the cardiovascular system but also capturing and disassembling the plaque in case of rupture. According to some embodiments, the device is preferably capable of one or more of treating vulnerable plaque, maintaining vessel patency, improving the structural integrity of devices such as stents, and/or preventing restenosis.

According to some embodiments, there is provided a device comprising a vessel support structure and at least one rotating portion supported by the vessel support structure. Optionally, an additional electric portion may obtain energy from the rotating portion. Optionally still, these components may optionally be integrated within one another producing a single unit comprising all three components. Optionally each of the components may be differentially attached or coupled to one another forming any number of combinations therefrom.

Optionally the rotating portion and the electric portion may be integrally formed within the vessel support structure. The rotating portion and electric portion may be optionally attached or coupled thereto either during manufacture, or after implanting the vessel supporting structure within a blood vessel.

The vessel support structure and the rotating portion of the device are optionally and preferably made of a dilatable and/or otherwise self-expanding structure that is optionally shaped according to the vessel, such as but not limited to a tubular structure, stent like structure, off the shelf (OTS) stent, or wire frame or other like vessel support structure. Similarly any of the embodiments of the present invention may be optionally implemented as a Stent-Graft that is optionally implanted in the aorta preferably in place of surgery. Various optional and preferable embodiments, use variations and implementations of this basic structure are presented herewith.

According to other embodiments, the present invention provides a device comprising at least one miniature rotating portion, optionally anchored to a vessel support structure used within the cardiovascular system. Optionally the device of the present invention may be implanted within the cardiovascular system, for example, in vessels such as the coronary arteries, aorta, carotid artery or any blood vessel or other bodily passageways having a flowing fluid.

Any of the embodiments of the present invention may optionally be deployed to a blood vessel. The deployment site may be optionally and preferably determined by any number of imaging methods known in the art and incorporated herein by reference for example including but not limited to X-ray fluoroscopy, intravascular ultrasound, echocardiography, MRI (magnetic resonance imaging), angioscopy, CT (computerized tomography) scan, and/or any other suitable imaging technology. Another optional mode of deployment is surgical, by direct insertion of the catheter carrying the device through a puncture of the targeted vessel in proximity to the deployment site.

Vessel support structures such as stents are implanted within blood vessels and are therefore subject and accommodate blood flow and circulation. Blood circulates in the human body due to a pressure gradient that drives the fluid from an area of high pressure to an area of relatively lower pressure. In the human body, blood circulates by the pumping action of the heart in a repetitive predictable manner, from the ventricles returning to the atria, due to the blood pressure gradient that exists between them. When the heart beats, the pressure in the arteries increases to a maximum, thereby creating the systolic pressure gradient that drives blood through the vessels. It is this pressure gradient, manifested in the blood stream that creates the hydrokinetic energy source that may be harnessed by the preferred embodiments of the present invention.

Preferably, the device comprising a rotating portion that, during lower diastolic blood pressure, continues to operate such that its functional ability is not limited by the level of pressure gradient. Furthermore, a preferred embodiment of the present invention features a rotating portion that is able to continue working for a period of at least a few seconds with zero blood flow due to the momentum of the rotating portion blades. This enables the rotating portion to work reliably regardless of the blood pressure phase.

The rotating portion according to a preferred non limiting embodiment of the present invention is able to harness the hydrokinetic energy of blood flow that causes the rotating portion blades to spin. The rotating portion of the preferred embodiment is preferably stably connected to a vessel support structure for example including but not limited to a tubular structure, stent like structure, wire frame or stent-graft or the like. Optionally, the rotating portion is stably connected to the frame of a vessel support structure in a number of optional configurations preferably including but not limited radialy within the vessel support structure, wherein the rotation portion is within the lumen of the vessel support structure. Optionally, the rotation portion may be coupled to the frame of the vessel structure wherein the rotating portion is preferably located extra-luminally, outside the lumen of the vessel support structure. Optionally, the rotating portion may be integrated within the plane of the vessel support structure's frame, preferably continuously integrated within the frame of the support structure.

The rotating portion preferably features a plurality of blades, optionally including but not limited to a two, three, four or five blade configuration. The rotating portion's blades optionally and preferably function also to clear the vessel support structure and its vicinity of any plaques that may have formed while optionally preventing any new plaques from reforming.

The vessel support structure and the rotating portion according to the preferred exemplary embodiment of the present invention is preferably expandable from a low-profile compressed condition to a larger profile expanded condition, wherein the resilient material urges the vessel supporting structure to expand radially, and to thereby apply radial force against the blood vessel's inner wall surface.

The preferred embodiments of the present invention comprising the vessel support structure and the rotating portion may optionally preferably be composed of a shape memory alloy (SMA) including but not limited to nickel titanium alloy (NiTi), also known as nitinol, having a transition temperature around body temperature. They may also (additionally or alternatively) optionally comprise a shape memory polymer (SMP) that can be triggered in response to changes in heat, pH, electric or magnetic fields. For example, the device of the preferred embodiment of the present invention may optionally be introduced into a blood vessel in its collapsed formation having a small profile. Once in place and after it is released from the constraining catheter, the device preferably expands to the appropriate diameter and into its final or “memorized” shape.

Optionally and preferably the rotating portion, according to a preferred embodiment of the present invention, is capable of axial flow as an axial flow rotating portion. More preferably, the rotating portion is an across-flow rotating portion. Of course any type of rotational direction may optionally be implemented. Such a rotating portion is preferably associated with the vessel support structure and is preferably smaller than the diameter of the vessel support structure's lumen. Optionally at least one rotating portion may be present within the diameter of the lumen. Optionally and preferably a plurality of rotating portions may be placed sequentially and incrementally, in a step like manner, to diagonally span the diameter of the vessel support structure. Optionally a plurality of rotating portions may so be placed wherein any cross section angle, including horizontally or vertically, is covered.

The optional, exemplary cross flow rotating portion preferably comprises a plurality of blades that are optionally shaped like a helical or skewback airfoil. These blades are optionally and preferably oriented transversely and perpendicularly to the fluid flow and parallel to the axis of rotation. Optionally, the blades have hydrofoil sections that provide tangential pulling forces in the cross fluid flow, allowing the forces to rotate the rotating portion in the direction of the leading edge of the blades. Therefore the direction of rotation of the rotating portion preferably depends significantly, and more preferably only, on blade orientation, rather than on the direction of fluid flow.

Preferably the blades of this embodiment of the present invention are helical airfoils that are warped into a spherical or elliptic shape. Optionally, the blades may have variable widths along the blade and/or its shaft. Optionally, different combination of blade types may be used an individual rotation portion. The orientation and shape of the rotating portion's blades preferably allow the rotating portion to be self-starting rotating portion, such that rotation is preferably initiated upon initiation of blood flow and such that the blades rotate even in very slow blood flow. Optionally, the shape and dimensions of the rotating portion may be controlled and adjusted to best accommodate one or more of vessel geometry and flow rate (and pressure). Optionally, the various components comprising the rotating portion, for example preferably including the blades and anchors, may be controlled and adjusted to best accommodate one or more of vessel geometry and flow rate (and pressure).

According to some embodiments, a change in the rotating portion activity and motion is monitored or detected. The detection may be optionally undertaken by imaging methods known in the art for example including but not limited to intravascular ultrasound, MRI (magnetic resonance imaging) or magnetic fluctuation sensor.

A still further optional non limiting embodiment provided by the present invention is an inherent blood flow rate sensor, where the blood flow rate passing through the device is proportional to the rotating portion rotation speed. Optionally, this may serve as either a primary or secondary sensor for devices associated with the rotating portion of the preferred embodiment of the present invention including but not limited to a implanted cardioverter defibrillator (ICD), potentially minimizing the defibrillator's false alarm rate. This exemplary non-limiting embodiment may optionally communicate vital data regarding the flow rate, cardiac output and vessel occlusion state as inferred from the activity or inactivity of the rotating portion. Optionally, the activity level of the rotating portion may be visualized by imaging methods known in the art, for example including, but not limited to intravascular ultrasound, MRI (magnetic resonance imaging) or magnetic fluctuation sensor

Optionally, the rotating motion and speed of the rotating portion may be controlled. Optionally rotation motion may be controlled from one or more external sources for example including but not limited magnetic energy induction. The blades of the rotating portion structure according to this optional embodiment may optionally comprise magnetic material. Optionally the blades may be composed of, or integrate, one or more permanent magnets. Alternatively, the blades could be produced from and/or coated with magnetic material.

In another embodiment, the rotating portion control is accomplished by placing one or more invasive endoluminal electrical cables connected to conductive windings incorporated within a vessel supporting structure. Such a configuration produces rotation of the rotating portion in a similar way to the rotation of electrical engine. Furthermore, according to this optional embodiment, such control and stimulus may optionally and preferably be used to recover the rotating portion operation in case of body tissues build-up which interfere with the rotating motion.

It should be noted that the rotating portion only harvests the hydrokinetic energy of the rotating portion sectional area through which the fluid passes. The shape, properties and/or number of blades of the rotating portion of the preferred non limiting embodiment of the present invention are optionally and preferably selected to provide the rotating portion with efficiency control.

Without limitation, it should be noted that one of the many distinct advantages of the preferred embodiments of the present invention is the ability to control the rotating portion operation. Optionally, control of the rotating portion may be gained by adjusting the blades' properties, for example according to one or more of blade shape, length, width, material or the like. This adjustment preferably allows the rotating portion to have a specific rotational efficiency and also more preferably allowing the fluid to flow freely, essentially not interfering with blood flow and not damaging the blood cells.

According to some embodiments, the blades are optionally and preferably mounted with at least one supporting member, which may optionally be mounted onto a rotatable shaft or a fixed shaft supported by at least one lightweight structure. The blades are preferably attached to an axis or shaft, and produce rotational spin about the axis or shaft, thereby creating rotational mechanical energy that may optionally be utilized to apply controlled force in the vicinity of the vulnerable plaque in order to intentionally crack the fibrous cap under controlled condition to induce a scar which is immune to future fissures or rupture (plaque sealing).

Optionally, the mechanical energy produced by the rotating portion of the present invention may be coupled to an electric portion to produce electrical, magnetic, optical and/or electrostatic energy. More preferably the electric portion may be integrated with the rotating portion of the present invention, thereby allowing the rotating portion blades to serve as a generator “rotor”; in this embodiment the “stator” windings are preferably incorporated within a vessel supporting. The electric portion windings are more preferably coated or covered with is electrical isolated material in order to isolate the generated electricity from the body. This optional configuration allows miniaturization of the rotating portion structure into miniature scale.

The electric portion in accordance with a further optional preferred embodiment of the present invention preferably features a generator module that converts and regulates the mechanical power to electrical power and an energy converter portion that converts the electrical power to one or more other forms of energy including but not limited to RF, heat and optical energy, most preferably as an healing and therapeutic energy.

The generator produces electrical energy optionally in the windings that are optionally and preferably coated with an electric isolating material. Optionally and preferably the produced electrical power may be used directly or indirectly optionally to treat the vulnerable plaque. The produced power may for example be used in the form of energy including but not limited to heat, direct electrical current, low level force, mechanical power, create scarring, or the like.

For example, it is believed that by applying certain procedures to treat the vulnerable plaque at a particular location, such treatment stabilizes the vulnerable plaque and/or prevents the plaque from proliferation or from further development towards rupture. The procedures include applying heat, delivering electrical charge, or by cracking the fibrous cap under controlled condition (micro-scar) and applying low level force in the vicinity of the unstable plaque using the mechanical power of the rotating portion.

Optionally treatment by heat may be undertaken in different ways, for example including but not limited to, coupling the device to an electrical load such as resistor or resistor-like element, electrical cables applying the electrical current directly, or utilizing infra-red or ultra-violet source such as LED to produce heating effect on surrounding tissues. Thus, the electrical current passing through the electrical load may optionally turn to heat.

In an embodiment, this is accomplished non-invasively by induction from an external induction source, such as described in U.S. Pat. No. 6,238,421 or EP 1,036,574, both of which are hereby incorporated by reference as if fully set forth herein. In another embodiment, this is accomplished using stent-like implanted structure, and heating of the implanted structure is a non-invasive inductive heating, such as described in U.S. Pat. No. 6,786,904 which is hereby incorporated by reference as if fully set forth herein.

According to an optional embodiment of the present invention, a device for the heating action is placed adjacent to the vulnerable plaque location or in the vicinity of the vulnerable plaque and is used to direct heating to adjacent tissue.

A further feature of some embodiments of the present invention is delivering electrical charge from the device to the lesion by positioning the device adjacent to the vulnerable plaque's lesion, thereby enabling the delivery of electrical charge from the device of the present invention to the vulnerable plaque lesion. Optionally, the device may be positioned adjacent to the vulnerable plaque and deliver the electric charge using an electric lead extender. Optionally the electrical charge delivery to the vulnerable plaque lesion, when applied for a sufficient time period, stimulates increased capillary growth near the lesion.

The device according to an optional embodiment of the present invention optionally stabilizes the vulnerable plaque by optionally providing therapeutic energy in the form or an infra-red (IR) light, ultra-violet(UV), electric charge, resistive load or other forms of energy that are projected to the vulnerable plaque area and/or other vascular areas of interest, more preferably in order to stimulate increased capillary growth near the vulnerable plaque lesion.

The vessel support structure and the rotating portion in accordance with a further optional preferred embodiment of the present invention may produce electrical energy (electric potential), or electrostatic energy by the addition of metals and/or polymers that are naturally charged, or, alternately, by incorporating piezo-electric materials which may generate electric potential. Such metals, polymers and piezo-electric materials may generate electricity or electric potential by the rotating action of the rotation device that generates electric potential.

In a further optional embodiment, the mechanical power of the rotating portion of the device of the present invention is preferably used to intentionally crack the fibrous cap under controlled condition to induce a scar which is immune to future fissures or rupture (plaque sealing). Thus once the acute effect with its inherent risk of acute occlusion has passed, the subsequent risk of plaque rupture is markedly reduced compared with that of an untreated plaque. Preferably the rotation portion utilized does not incorporate power generator capabilities.

Optionally any of the embodiments used for the purpose of treating a vulnerable plaque may be used in combination to preferably further accelerate the stabilization process of the vulnerable plaque, thereby stimulating the body to generate additional layer of calcified cap and therefore building a thicker calcified cap.

According to other embodiments, optionally the rotating portion is coupled to a filtering portion to preferably capture and disassemble ruptured plaque that breaks off from the vessel. Optionally the blood clot filtering portion is deployed between the rotating portion blades and is fixedly coupled to the rotating portion, and thereby preferably rotates together with the rotating portion. The filtering portion is preferably formed from a plurality of elongated strands arranged to form a general filtering structure. n a further optional embodiment, the filter portion may optionally be formed as a net for filtering the particulate matter that typically form a basket-like conic structure as already widely used. For example, the filter portion may optionally be manufactured from a flexible thread such as surgical monofilament sutures suitable for insertion into the body and/or for medical use. Other materials may optionally be used; for example, metallic material, such as titanium, gold, and/or suitable alloys may optionally be used.

An optional embodiment of the present invention is optionally used to treat vulnerable plaques by using a device of the present invention optionally comprising vessel support structure, and preferably comprising at least one rotating portion and a generator. Preferably the vessel supporting structure is expandable from a low-profile compressed condition to a larger profile in the expanded condition. Preferably the vessel supporting structure is anchored to the inner walls of a vessel in the vicinity of a vulnerable plaque.

A device or one of its components according to any of the embodiments of the present invention may optionally be used either temporarily for acute medical procedures or permanently for long term treatment.

Optionally, the vessel support structure of the present embodiment may be positioned in a temporary or retrievable manner while being positioned adjacent or in the vicinity of a vulnerable plaque. Preferably the device of the present invention is stationary for the duration of a treatment period and is preferably retrieved after treatment by medical instrumentation, more preferably including but not limited to an introducer catheter that may be re-advanced over the guidewire to a site of the device assembly in order to retrieve the device.

The guidewire may optionally be retracted proximally such that the locking mechanism of the guidewire interacts with the vessel supporting structure or the rotating portion mechanism of the device, and pulls the device proximally either partially or completely into the introducer catheter. The device is then removed from the vasculature.

Alternatively, for example, following the completion of the therapeutic or diagnostic procedure, an angioplasty catheter with distal introducer may optionally be used to retrieve the device by withdrawing the guidewire proximally into the distal introducer and pulling the device into the distal introducer. Alternatively, during short term implementation the device in any one of its embodiments may optionally be placed in the vasculature while it is optionally kept in place by being attached to the distal end of an elongated member, for example including but not limited to a guidewire, such that once the procedure is concluded, optionally the device in any one of its embodiments of the present invention may be retrieved.

A further feature of the present invention in any one of its embodiments is its temporary use wherein preferably only the rotating portion and/or the electric portion are retrieved by medical instrument or intervention, for example including but not limited to an angioplasty catheter with distal introducer, for example as described herein.

A further exemplary embodiment preferably features a non-compliant balloon that can be inflated against the vessel walls in order to spread the luminal components such as the rotating portion against the vessel wall. Optionally the rotating portion and/or the electric portion may be securely attached and removed from the vessel support structure, preferably with a mechanical assembly such as but not limited to turn lock assembly or a pin lock assembly and more preferably by taking advantage of the device's material properties that can optionally be triggered in response to changes in heat, cold, pH, electric or magnetic fields in the vicinity of the rotating portion and the electric portion applied by external source. Optionally the rotating portion and/or the electric portion may be controllably released with any combination thereof.

Optionally the device vessel support structure is shaped as an airfoil on internal surfaces of the vessel support structure for increasing blood flow through the rotating portion. Optionally and preferably, the increased blood flow through the rotating portion facilitates the treatment of atherosclerosis and especially a vulnerable plaque.

Optionally for the device of the present invention in any of its embodiments of the present invention comprising the vessel support structure, the rotating portion is preferably composed of metallic material for example including but not limited to 300 series stainless steels, platinum, platinum-iridium alloys, cobalt-chromium alloys such as MP35N, unalloyed titanium, or the like.

The device of the present invention in any of its embodiments may optionally be coupled to various objects and therefore may serve as a platform for introducing secondary objects for example including but not limited to sensors, medicaments, small scale devices, or the like. Most preferably the secondary objects are coupled to the rotating portion of the device of the present invention preferably utilizing the rotational speed to control the secondary device. For example, the rotational speed of the rotating portion may optionally and preferably act as a trigger to release the medicament, electrical current, or to communicate sensed data for example blood pressure or the like.

Optionally the device of the present invention, in any of its embodiments, may be coupled to the secondary object by various means that are conducive both to the secondary object and the device itself, for example coupling according to a method including but not limited to loading, or coating or the like.

For example, the device of the present invention, in any of its embodiments, may optionally serve as a platform for carrying at least one sensor for example including but not limited to physiologic sensor, hematological sensor, biochemical sensor and like sensors. Preferably and optionally a sensor may be coupled to the rotating portion of the device of the present invention. The sensor optionally and preferably enables continued monitoring of at least one parameter for example including but not limited to temperature, blood pressure, heart rhythm, pH, electrolytes, blood sugar, blood cholesterol levels or the like.

The device of the present invention in any of its embodiments may be coupled with a medicament for example including but not limited to blood thinning drugs, hormones, genes or the like, through a medicament dispenser. Most preferably the medicament dispenser is coupled to the rotating portion of the device. Optionally the device may be loaded with a medicament, for example by coating or by coupling small aggregates that release the medicament, for example including but not limited to hormones, genes or the like, in which said coating and/or said small aggregates optionally comprise an optional type of dispenser. Optionally the release of the medicament may preferably be controlled by the activity of the rotating portion of the device and may optionally include automatic release, uniform release, or any type of reaction including but not limited to a magnetic reaction, rotating speed reaction (high or low blood rate), servomechanism, sensor programming or external control.

In any some of the embodiments of the present invention, the device may act as a foreign body that may stimulate the formation of thrombi or occluding material. Accordingly, the deployment of the device of the present invention may be coupled to a course of drug treatment, for example including but not limited to drugs that inhibit or control the formation of thrombus or thrombolytics such as heparin or heparin fragments, aspirin, coumadin, tissue plasminogen activator (TPA), urokinase, hirudin, and streptokinase, and other suitable therapies may be used.

The device of the present invention in any one of its embodiments may be optionally coated with a substance or structure that improves biocompatibility or tissue adaptation or coated for in vivo compatibility as is described and well known in the art. The device may be coated with one or more of the following: antiproliferatives (methotrexate, cisplatin, fluorouracil, Adriamycin, antioxidants (ascorbic acid, carotene, B, vitamin E, and the like), antimetabolites, thromboxane inhibitors, non-steroidal and steroidal anti-inflammatory drugs, Beta and Calcium channel blockers, genetic materials including DNA and RNA fragments, and complete expression genes, carbohydrates, and proteins including but not limited to antibodies (monoclonal or polyclonal) lymphokines, growth factors, prostaglandins, and leukotrienes, and other suitable therapies may be used.

The device of the present invention in any one of its embodiments may optionally in whole or in part be formulated or composed of biodegradable material, preferably reducing the risk of thrombus formation and reducing need for chronic implantation of vessel support structures. Optionally the biodegradable material may be composed of materials such as polylactic acid polyglycolic acid (PGA), collagen or other connective proteins, magnesium alloys, polycaprolactone, hylauric acid, adhesive proteins, co-polymers of these materials as well as composites and combinations thereof and combinations of other biodegradable polymers, biodegradable glass, bioactive glass or the like. Preferably, biodegradable glass and/or bioactive glass are also a suitable biodegradable material for use in the present invention.

For example, the vessel support portion of the device may optionally be composed of biodegradable material wherein preferably over time the support structure is absorbed by the body after healing of the angioplasty site, thereby alleviating the need for chronic implantation. Optionally and preferably it would be further desirable to use biodegradable material that could be shaped in a desirable manner for example including but not limited to a mesh-like or porous configuration, that will optionally enable endothelial cells at the angioplasty site to grow into and over the vessel supporting structure so that bio-degradation will occur within the vessel wall. Similarly, any portion of the device may optionally be formed with biodegradable matter for example including but not limited to the rotation portion, that may optionally be degraded over time. Degradation may optionally (additionally or alternatively) be triggered by a triggering event such that the biodegradation will preferably start for example when the site is deemed to be healed. For example, in some implementations, such as stent or stent-graft or an in-stent restenosis (ISA) the rotating portion of the device may preferably not be required beyond a certain period of time. Optionally in such implementation the rotating portion may comprise biodegradable material to minimize the risk of thrombus formation and other complications.

Also it should be emphasized that although the described embodiments refer to blood vessels and blood flow, the present invention may also optionally be employed within other lumen(s) of the body, preferably those lumen(s) through which there is fluid flow, including but not limited to the cerebrospinal fluid system, the lymphatic system, the gastrointestinal tract, the kidneys and bladder (urinary tract and associated system), the male reproductive tract, fluid flow within the eyes and so forth. In such a context, plaque may optionally be interpreted to include any type of blockage or stricture.

Unless otherwise defined, all technical and scientific terms used herein have the same meaning as commonly understood by one of ordinary skill in the art to which this invention belongs. The materials, methods, and examples provided herein are illustrative only and not intended to be limiting. Implementation of the method and system of the present invention involves performing or completing certain selected tasks or steps manually, automatically, or a combination thereof.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1A-C are schematic diagrams of an exemplary miniature rotating portion according to an optional embodiment of the present invention;

FIG. 2A-C are schematic diagrams of an exemplary miniature rotating portion according to an optional embodiment of the present invention;

FIG. 3A-C are schematic diagrams of an exemplary miniature rotating portion according to an optional preferred embodiment of the present invention;

FIG. 4 is a schematic diagram of an exemplary mounting of the miniature rotating portion according to an optional embodiment of the present invention;

FIG. 5A-H are exemplary schematic diagrams of the blades used in the rotating portion according to an exemplary embodiment of the present invention;

FIG. 6A-G is an exemplary schematic diagram of the vessel supporting structure and the rotating portion according to an exemplary embodiment of the present invention as implanted in a blood vessel;

FIGS. 7A-C and 8A-C depict an alternative optional mode of delivery of the preferred embodiment of the present invention

FIG. 9 depicts an optional mode of device delivery and realigning of the preferred embodiment of the present invention; and

FIG. 10A is an exemplary schematic diagram of an exemplary embodiment of the present invention for treating vulnerable plaque using optic and electric energy transmission; and

FIG. 10B-D depict different views of an exemplary device used to treat a vulnerable plaque using optic and electric energy transmission; and

FIG. 11A-C are exemplary schematic diagrams of exemplary embodiments of the present invention based on rotating portion placement.

FIG. 12A-C are exemplary schematic diagrams of different views of placement of a plurality of rotating portion according to an exemplary embodiment of the present invention; and

FIG. 13 depicts a schematic diagram of a rotating portion placed adjacent to a vulnerable plaque according to an optional embodiment of the present invention; and

FIG. 14A-B are schematic diagrams of a rotating portion within a wire frame placed adjacent to a vulnerable plaque according to an optional embodiment of the present invention; and

FIG. 15A-D are schematic diagrams of the delivery catheter of the device according to a preferably embodiment of the present invention; and

FIG. 16 is a block diagram of the energy generating and conversion process according to present invention.

### DESCRIPTION OF THE PREFERRED EMBODIMENTS

The present invention is of a system and a method for a hydrokinetic rotating portion that is implanted in a blood vessel, preferably within a vessel support structure, including but not limited to a tubular structure, stent like structure, wire frame, stent-graft or the like. The rotating portion may be coupled to electric portion having variable uses, including but not limited to one or more of generating power and treating vulnerable plaque.

The principles of the present invention may be better understood with reference to the drawings and the accompanying description.

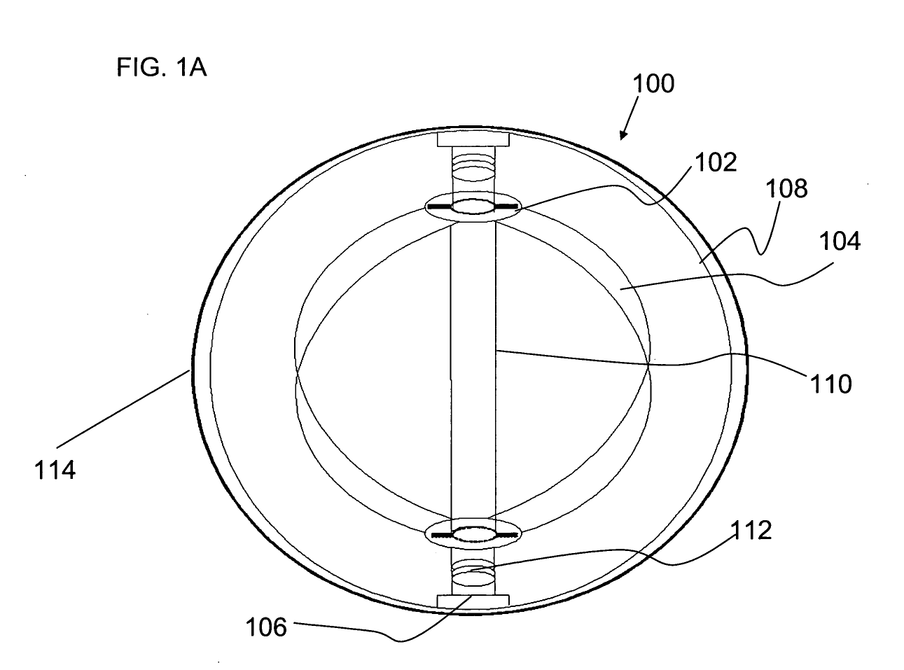

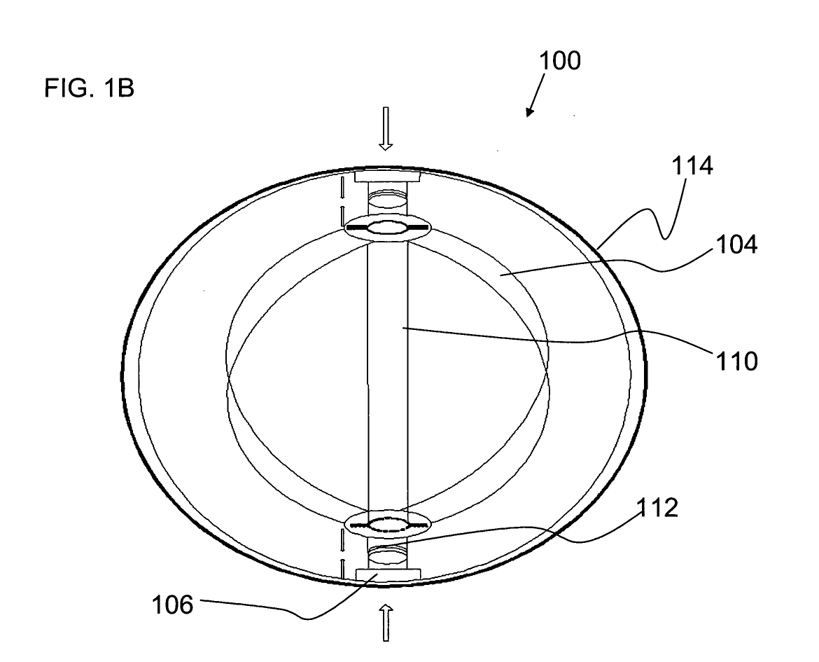

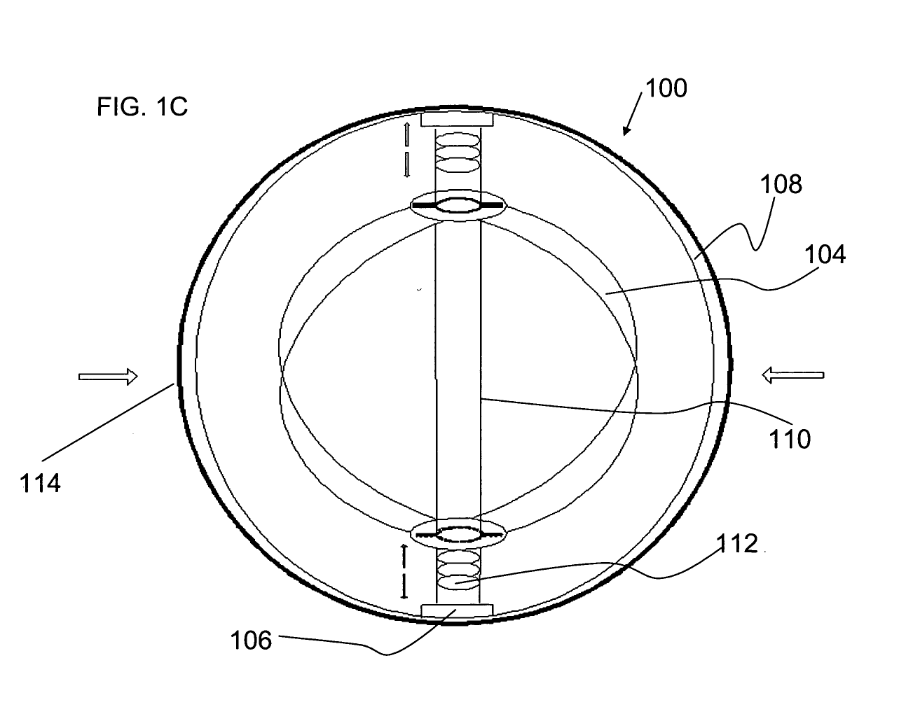

Referring now to the drawings, FIG. 1A is a schematic cross section diagram of an exemplary rotating portion 100 according to an optional embodiment of the present invention. Rotating portion 100 is attached to a vessel support structure 108, preferably including but not limited to a tubular structure, a stent, blood clot filter, wire frame and stent-graft. Vessel support structure 108 is inserted within the lumen of a blood vessel wall 114, preferably according to the stent introducing techniques known in the art, and incorporated herein by reference, utilizing a guiding catheter, a guide wire and a balloon angioplasty catheter. The stent delivery system that includes the stent is optionally and preferably advanced over the guide wire and the stent is then deployed at the site of the dilated stenosis (not shown).

Once in place within the blood vessel walls 114, the rotating portion 100 keeps the lumen open allowing blood to flow therethrough. Furthermore the movement of blades 104 preferably captures and disassembles any plaque or occluding material flowing through or building up in the lumen of vessel support structure 108. A rotating portion axis 110 preferably spans the diameter of the vessel walls 114, and is preferably positioned transversely and perpendicularly to the direction of blood flow to produce rotation in a plane parallel to the direction of blood flow. Rotating portion 100 is preferably anchored to vessel support structure 108 by at least one anchor 106. Blades 104 are preferably coupled to rotating portion axis 110 through rotors 102 which are optionally molded or otherwise integrally formed with blade 104 to create a uniform structure. Blades 104 are optionally shaped to maximize rotational speed and/or to provide a specific energy generation efficiency. Preferably blades 104 are helical and airfoil-shaped, and spherically or elliptically warped. As the blood flows through the lumen of the vessel support structure 108, it causes blades 104 and rotors 102 to spin. It is the spinning blades 104 and rotors 102 that define the operation of rotating portion 100 with respect to maintaining the lumen of vessel 114 free of potential blockages. Furthermore, anchor 106 increases overall stability of vessel support structure 108 and vessel walls 114.

However, because vessel walls 114 are not static and immobile, rotating portion 100 and vessel support structure 108 are preferably sufficiently flexible to accommodate for the natural forces acting on vessel walls 114. Accordingly, at least one flexible support structure 112, optionally including but not limited to a spring like element, is integrated between axis 110 and anchor 106, that provide rotating portion 100 with the required flexibility, allowing it to size itself according to the changing shape of vessel walls 114 while providing support structure 108 with sufficient structural integrity.

An optional but preferred extent of flexibility of rotating portion 100 is shown in FIGS. 1b-c. Accordingly, rotating portion structure 100 is preferably able to adjust one or more dimensions in accordance with the size of blood vessel walls 114. Flexible support structure 112 and flexible blades 104 preferably provide the structure of rotating portion 100 with the desired flexibility, allowing the rotating portion 100 to reshape itself in accordance with the various forces acting on the vessel walls 114.

FIG. 1B is an exemplary depiction of how rotating portion 100 and vessel support structure 108 may optionally and preferably be reshaped (i.e. to have a change in at least one dimension) in accordance with vertical forces that act on the vessel walls 114. Flexible structure 112 and flexible blades 104 absorb the applied vertical force, and preferably adjust at least one dimension to condense and hence to reshape rotating portion 100 to appropriately fit vessel walls 114.

Similarly, FIG. 1C is an exemplary depiction of how rotating portion 100 may optionally and preferably be reshaped (i.e. to have a change in at least one dimension) in response to horizontal forces acting on vessel walls 114. Flexible support structure 112 and flexible blades 104 preferably absorb the applied vertical force and expand, thereby adjusting at least one dimension to reshape rotating portion 100 to better fit vessel walls 114. Thus, blades 104, optionally made of pliable material, also preferably change configuration to better fit vessel walls 114 as constriction forces are applied on blades 104 both vertically and horizontally. Rotating portion 100 is optionally made of pliable material, allowing rotating portion 100 to be reshaped numerous times.

Blades 104 are optionally and preferably made of pliable material, optionally including but not limited to magnetic material incorporated within the blades 104, and/or a magnetic coating on the outer surface of blade 104 which may optionally be used to generate electrical power. Optionally blades 104 and rotors 102 may be fitted with other energy harvesting materials and devices that would allow use of their rotational energy to create alternative forms of energy.

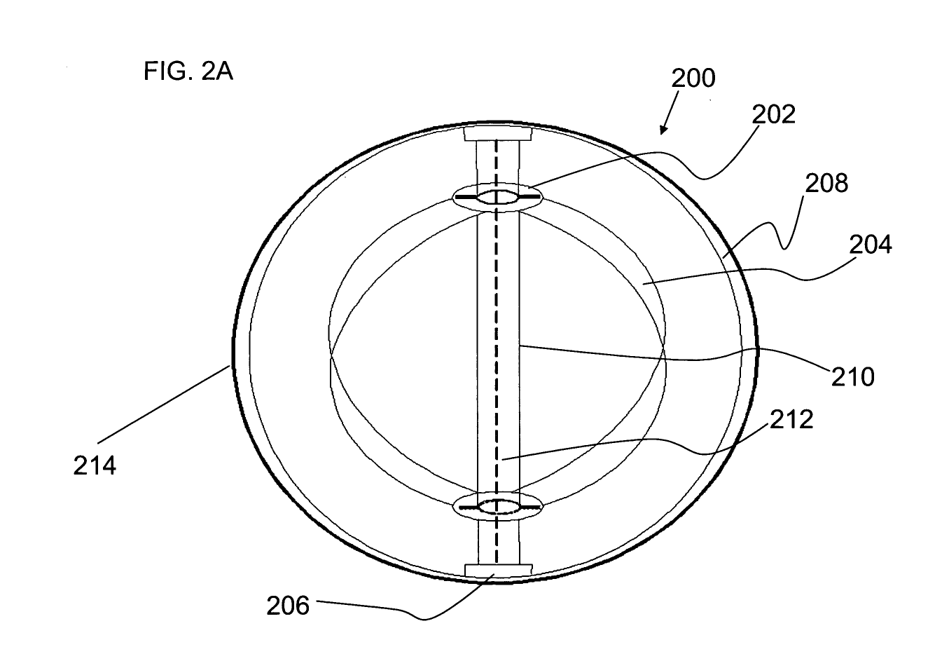

An additional embodiment of rotating portion 100 of FIG. 1 is shown in FIG. 2 that shows an optional, exemplary embodiment of the present invention. A rotating portion 200 is implanted within the lumen of a blood vessel wall 214, and as described above that is supported by a vessel support structure 208, preferably including but not limited to a tubular structure, a stent, blood clot filter, wire frame and stent-graft. A rotating portion axis 210 is preferably made of pliable or flexible material, and more preferably spans the diameter of the vessel support structure 208 lumen. Rotating portion axis 210 is also preferably positioned transversely to the direction of fluid flow, producing rotation in a plane parallel to the direction of fluid flow. Rotating portion axis 210 is optionally and preferably held in position relative to vessel support structure 208 by at least one anchor 206. Along rotating portion axis 210 at least one and more preferably two rotors 202 are optionally molded with and/or integrally formed with at least one blade 204, that is optionally an airfoil-shaped spherically warped blade and/or a skewback type blade. As blood flows through the lumen of vessel support structure 208, it applies rotational forces on blades 204, causing them to spin and in turn spinning rotors 202. Axis 210 is preferably made from pliable material and provides rotating portion 200 with flexibility, allowing it to size itself (i.e. to change at least one dimension) according to the changing shape of vessel walls 214, as depicted in FIGS. 2B-C and also as noted previously with regard to FIG. 1.

Vessel walls 214 are not static, such that their shape is modified as constrictive forces are applied on them. Accordingly, rotating portion structure 200 is preferably able to resize itself (i.e. to change at least one dimension) in accordance with the size of blood vessel walls 214. Axis 210 preferably provides rotating portion structure 200 with the required flexibility and structure for such alteration of at least one dimension.

FIG. 2B depicts how the rotating portion may optionally and preferably reshape (i.e. to change at least one dimension) in accordance with vertical forces that act on the vessel walls 214. Axis 210 preferably absorbs the applied vertical force and condenses to reshape device 200 to fit vessel 214.

Similarly, FIG. 2C is an exemplary depiction of how rotating portion 200 may optionally and preferably reshape (i.e. to change at least one dimension) in response to horizontal forces acting on vessel walls 214. Axis 210 preferably absorbs the applied vertical force and expands to reshape rotating portion 200 to fit vessel walls 214 reconfigured shape. Furthermore, blades 204, optionally molded and/or integrally formed with rotors 202, are optionally made of pliable material and able to change configuration to fit the new shape with the vessel walls 214 as constriction forces apply to it both vertically and horizontally. Preferably rotating portion 200 is optionally made of malleable and pliable material allowing it to be continuously reshaped.

Blades 204 are optionally made of pliable material, including but not limited to a magnetic material incorporated within the blades and/or magnetic coating on the outside of blades 204, that optionally functions to generate electric charge. Blades 204 and rotors 202 may optionally generate an electric charge in order to further prevent blood components from collecting optionally incorporating metals and/or polymers that are naturally charged, or by incorporating piezo-electric materials which may generate electric potential that generate electric charge by the rotating action.

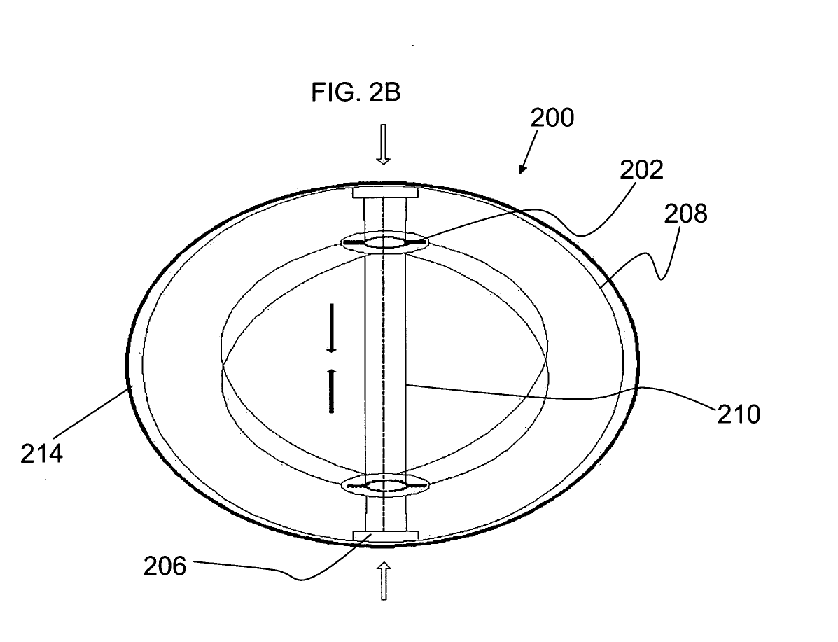

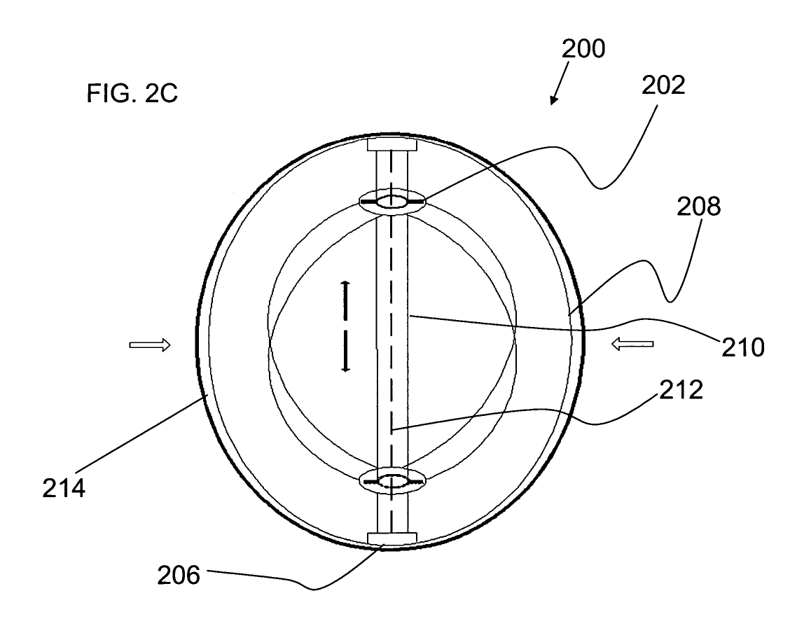

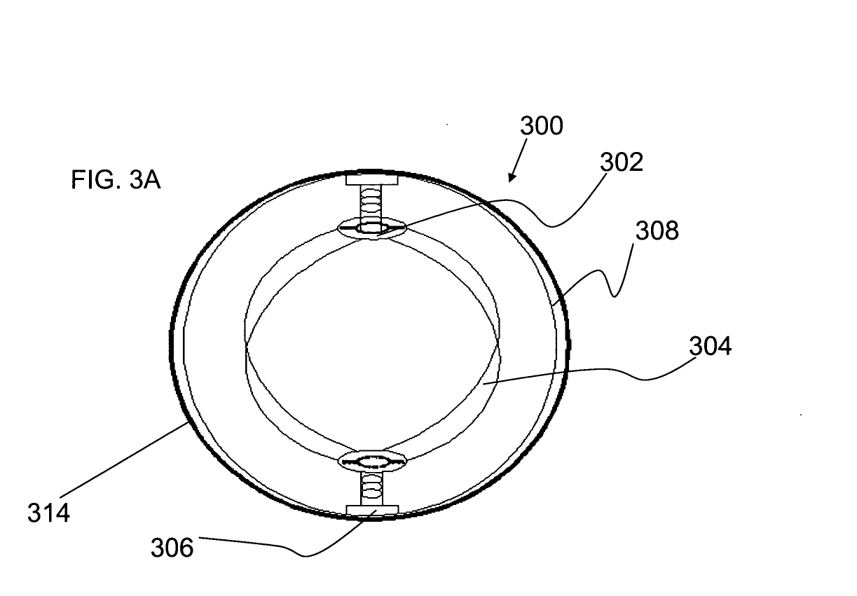

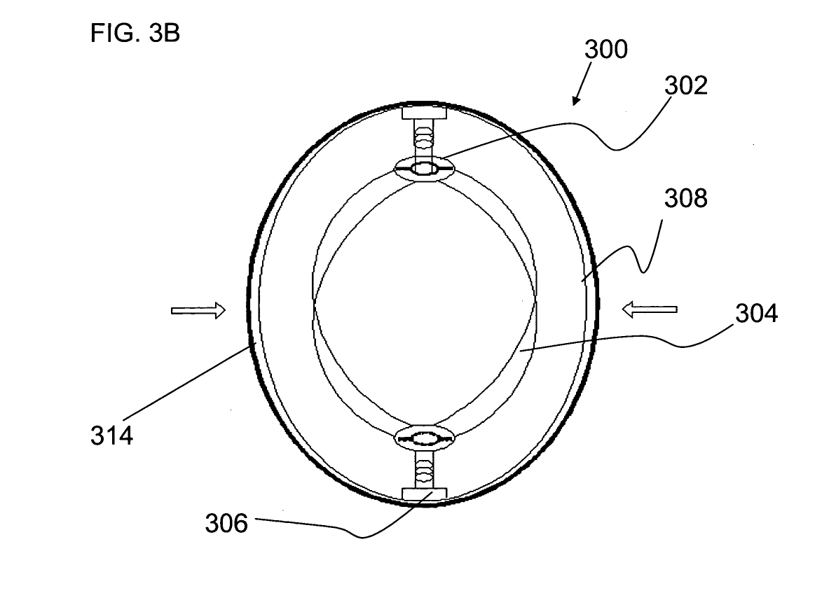

FIG. 3 presents a still further optional embodiment of the present invention that is another optional configuration of the rotating portion structure introduced in FIGS. 1 and 2 above. A rotating portion 300 is preferably implanted within the lumen of a blood vessel wall 314 supported by a vessel supporting structure 308, optionally including but not limited to a tubular structure, a stent, a coated stent, blood clot filter, wire frame and stent-graft. At least one support anchor 306 is preferably positioned transversely to the direction of fluid flow to produce rotation in a plane parallel to the direction of blood flow, while securing rotating portion 300 in position relative to vessel support structure 308. At least one and preferably two rotors 302 are positioned and optionally molded and/or integrally formed with at least one blade 304, which is optionally and preferably helical and airfoil-shaped, and is more preferably spherically warped. As blood flows through the lumen of vessel support structure 308, it applies a force on blades 304 causing them to spin, and in turn this causes rotors 302 to spin. Blade 304, preferably made of pliable material, provides rotating portion 300 with flexibility allowing it to size itself (i.e. to change at least one dimension) according to the changing shape of vessel walls 314.

FIG. 3B is an exemplary depiction of how rotating portion 300 may reshape in response to vertical forces that act on the vessel walls 314. Anchors 306 preferably absorb the applied vertical force and condense (i.e. to change at least one dimension) to reshape rotating portion 300 to fit vessel 314.

Similarly, FIG. 3C exemplary depicts how rotating portion 300 may optionally and preferably reshape in accordance with horizontal forces acting on vessel walls 314. Anchors 306 preferably absorb the applied vertical force and expand to reshape (i.e. to change at least one dimension of) rotating portion 300 to fit a reconfigured shape of vessel walls 314. Furthermore, blades 304 preferably change configuration to fit the new shape with the vessel walls 314 as constriction forces act on blades 304 both vertically and horizontally. Preferably rotating portion 300 is optionally made of malleable and pliable material allowing it to be repeatedly reshaped.

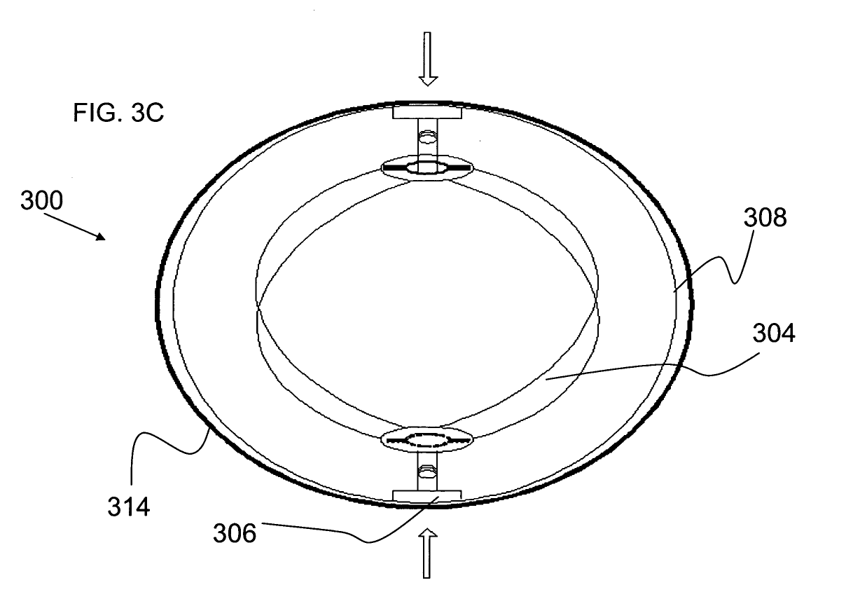

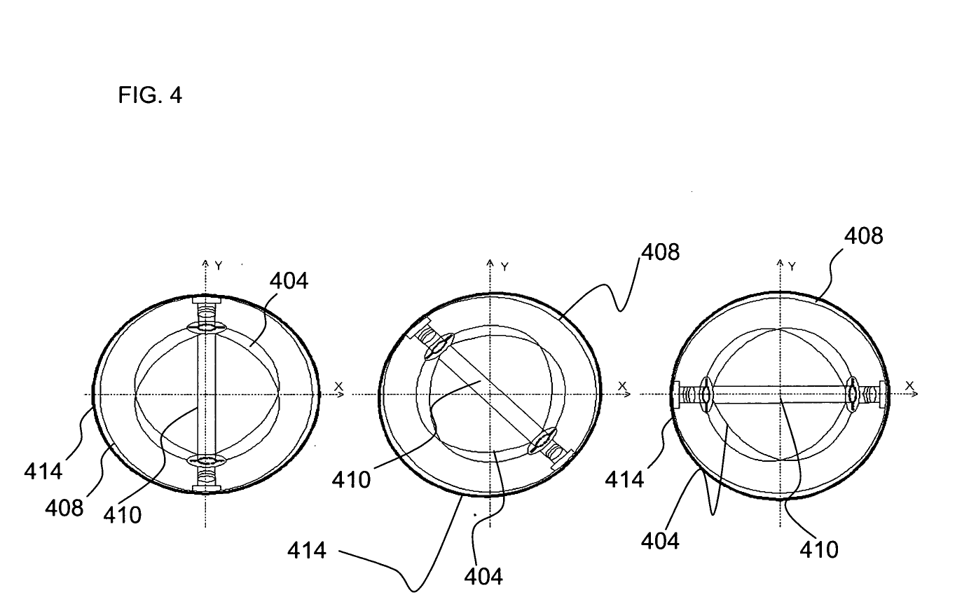

FIG. 4 shows an optional non-limiting embodiment of rotating portion, with regard to mounting of the embodiment described in FIG. 1 above, such that rotating portion axis 110 (of FIG. 1) has been rotated 90 degrees to produce rotating portion axis 410 having a horizontal orientation. This rotation shows that a rotating portion according to any one of the embodiments of the present invention may be oriented in any manner within the vessel supporting structure 408 and blood vessel walls 414 as the shape of blade 404 determines the rotational direction.

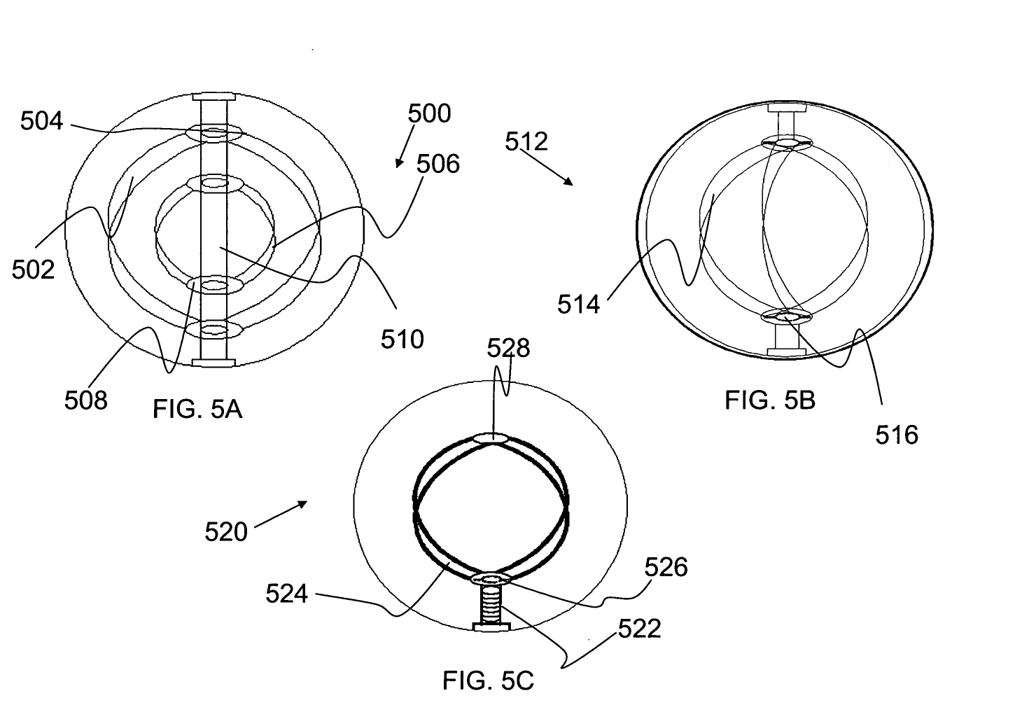

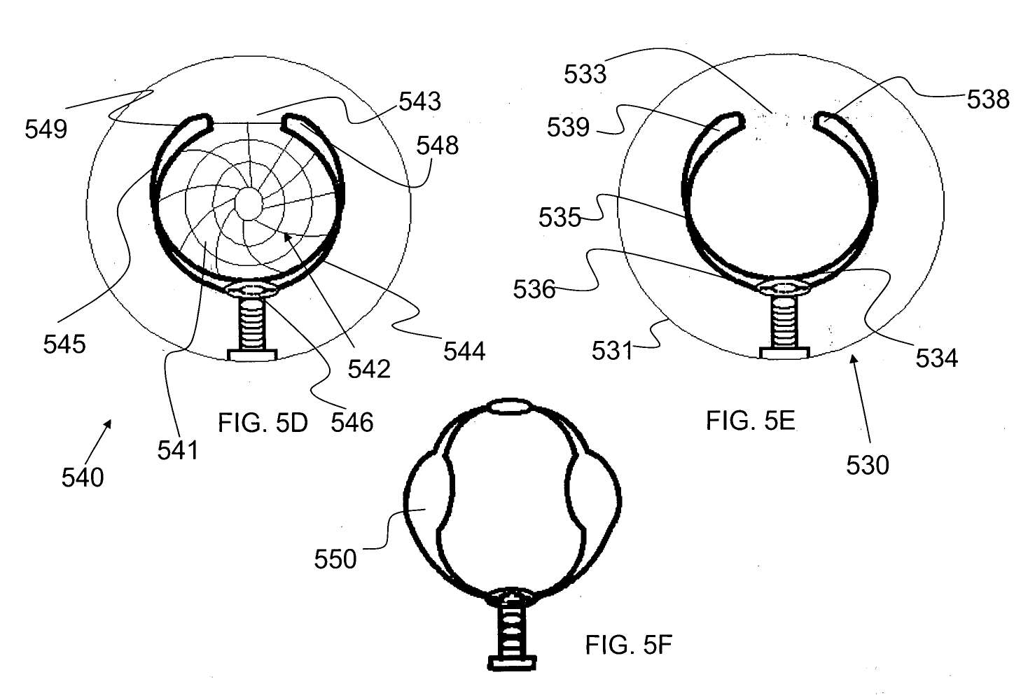

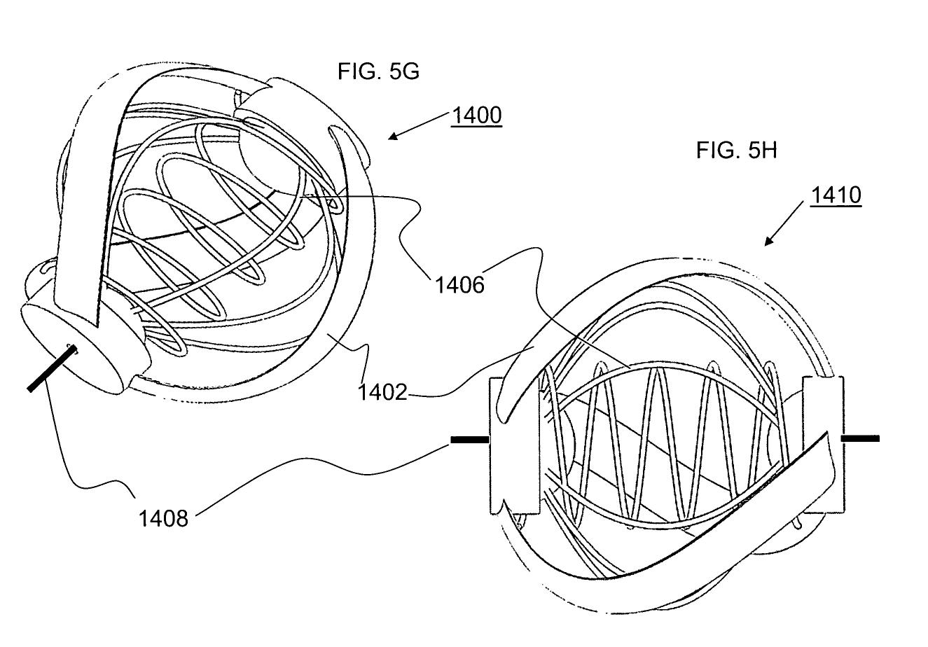

FIG. 5A is a depiction of a still further non limiting embodiment of the blades of the present invention, having an exemplary multiple layer configuration 500. Multiple layer configuration 500 preferably comprises at least two concentric blades as shown, an inner blade 506 attached to axis 510 via rotors 508 (optionally molded and/or integrally formed with inner blade 506), and an outer blade 502 optionally molded and/or integrally formed with rotors 504. The adjacent blades, inner blades 502 and outer blades 506, are preferably shifted circumferentially such that they do not overlap each other during rotation. That is, inner blades 506 preferably generate a spherical shaped rotating portion, which is positioned inside the outer spherical shaped rotating portion. The radius of inner blades 506 is preferably always smaller than the radius of the outer blades 502. The multilayer arrangement increases the torque of rotating portion 500.

FIG. 5B is a depiction of a still further non limiting embodiment of the rotating portion blades having a triple blade configuration 512. Triple blade configuration 512 preferably has three blades 514 that are optionally molded with rotor 516. The triple blade configuration may optionally be implemented with any rotating portion assembly configuration of a non-limiting embodiment of the present invention. Other multiple blade configurations having a plurality of blade groupings may optionally and preferably be implemented within the present invention.

FIG. 5C is a depiction of a still further non limiting embodiment of the rotating portion blades having one anchor configuration 520. Single anchor configuration 520 comprises one flexible anchor 522 and one rotor 526 that is optionally molded and/or integrally formed with at least one blade 524. Optionally, blade 524 is fitted with a flexible attachment 528 allowing a plurality of blades to be connected thereto. Anchor configuration 520 may optionally be implemented with any of the rotating portion assembly configurations previously presented according to the non-limiting embodiments of the present invention.

FIG. 5D is a depiction of a still further optional non limiting embodiment of the rotating portion blades having one anchor and an open configuration with a filter assembly 540. Filter assembly 540 preferably features a rotor 546 which is positioned and optionally molded and/or integrally formed with at least one blade 544. The blade upper tip 548 is open and is preferably rounded to prevent damaging the vessel walls. There is preferably a safety gap 543 from upper tip 549 of the other blade 545, which is preferably present to prevent surgical equipment, including but not limited to a guide wire, balloon angioplasty catheter and stent delivery system, from being caught between the upper tips 548 and 549 of the rotating portion blades 544 and 545, respectively. The blood clot filtering portion 542 is preferably deployed between the rotating portion blades 544 and is more preferably coupled thereto.

Filtering portion 542 is optionally formed from a plurality of elongated strands 541, optionally arranged to form a net or web-like structure in order to catch and hold blood clot(s) and/or other material or debris that are flowing in the blood stream. Elongated strands 541 are optionally and preferably fixedly attached to one another only at the apex of the filtering portion 542. As known in the art, the elongated strands 541 may optionally be formed from metallic material such as titanium and nitinol (nickel-titanium alloy), plastically deformable material, temperature-sensitive shape memory material with a transition temperature around body temperature, flexible thread such as surgical monofilament sutures or any elastic material preferably having a core formed from radiopaque material suitable for insertion into the body. Filter assembly 540 may optionally be implemented with any rotating portion assembly configuration of a non-limiting embodiment of the present invention.

FIG. 5E is a depiction of a still further optional and non limiting embodiment of the rotating portion blades having a single anchor open configuration 530. Single anchor open configuration 530 preferably comprises one rotor 536 optionally molded and/or integrally formed with at least one blade 534. Upper tip 538 of blade 534 is open and preferably rounded to prevent damage to vessel wall 531, more preferably having a safety gap 533 from the upper tip 539 of another blade 535, for example to prevent a balloon angioplasty catheter from being caught between upper tips 538 and 539 of the rotating portion blades 534 and 535. The one anchor open configuration 530 may optionally be implemented with any rotating portion assembly configuration of a non-limiting embodiment of the present invention.

FIG. 5F is a depiction of a still further optional non limiting embodiment of the rotating portion's blades 550 having variable width along its length. Preferably the blades may be designed to produce the required rotational speed and momentum.

FIGS. 5G and 5H depict two planar views of some embodiments of the rotating portion of the device according to the present invention. Rotational portion 1400 and 1410 comprises blades 1402; wire mesh structure 1406; and anchor structure 1408. Anchor structure 1408 optionally and preferably may be coupled to various devices including but not limited to a vessel support structure (not shown), a catheter (not shown), guidewire (not shown) or the like. For example a vessel support structure (not shown) may be optionally coupled to rotating portion 1400 or 1410 for a long term care application where a vessel support structure is implanted within a vessel.

Optionally wire mesh structure 1406 may undertake various functions for example including but not limited to a filter that traps particles in the flow or optionally as a windings that interact with blades 1402 that are optionally coated with a magnetic substance and/or otherwise feature a magnetic substance, to preferably generate electric charge. The rotation of rotating portion 1400 and 1410 may optionally also be one or more of controlled, produced, enhanced and/or accelerated externally by induction from an non-invasive external induction source for example including but not limited to: magnetic energy source, and/or by placing an invasive endoluminal electrical cable, connected to the conductive windings incorporated within a vessel support structure. Thus, accelerating the rotation of the rotating portion in achieved in a similar manner as an electrical engine.

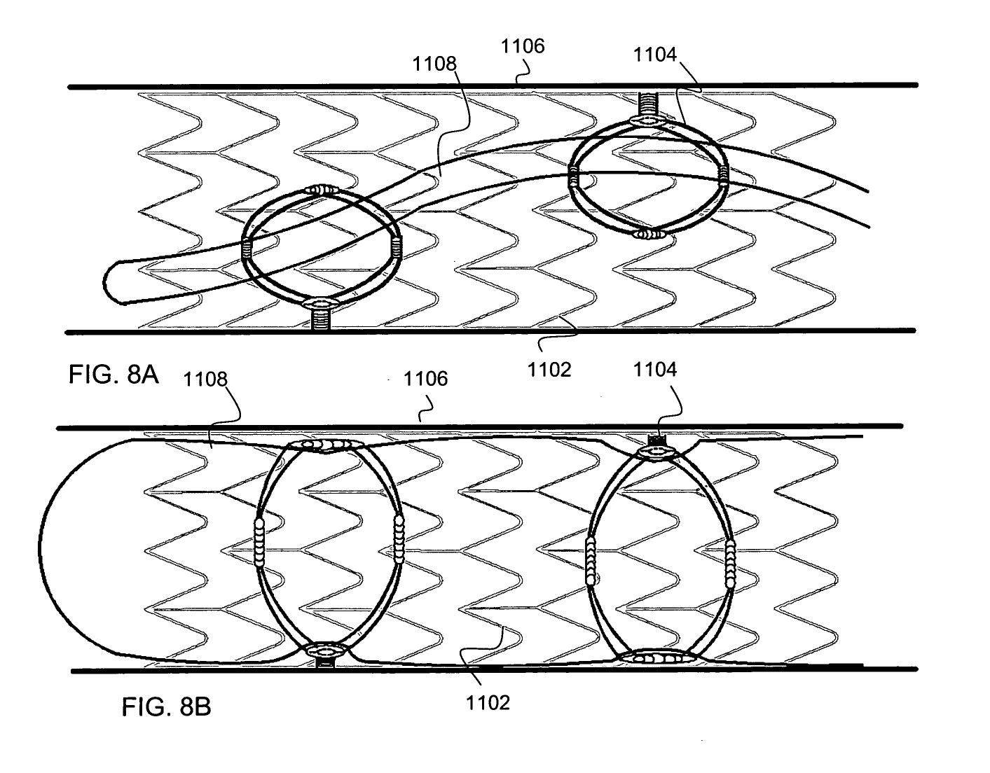

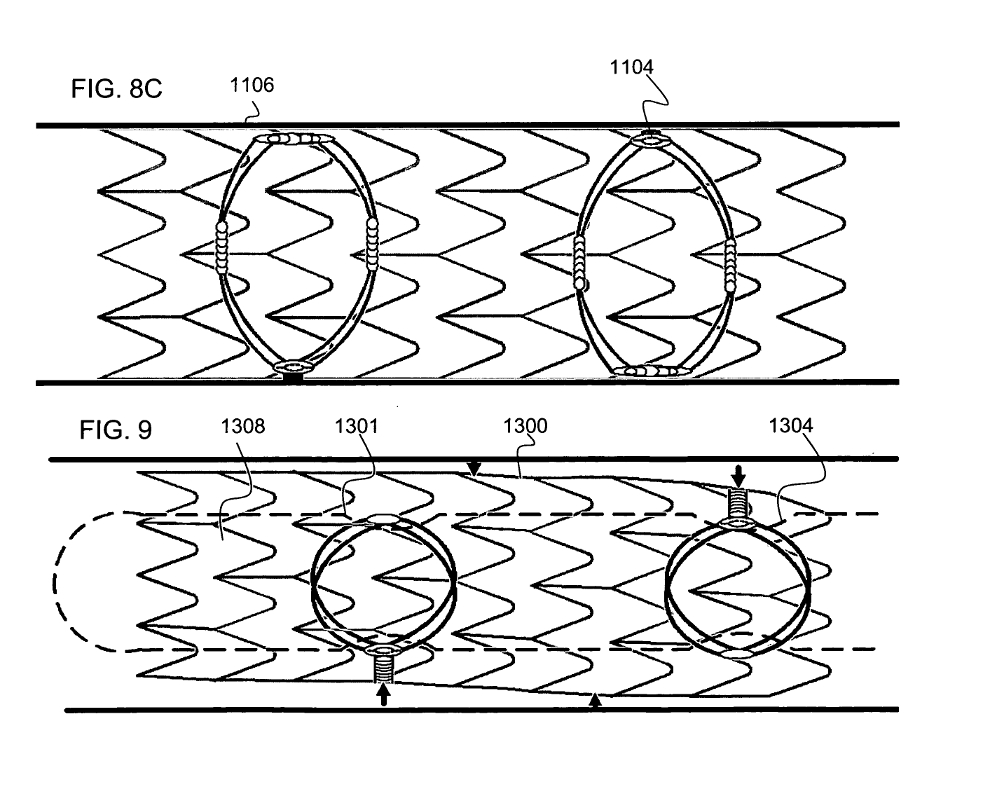

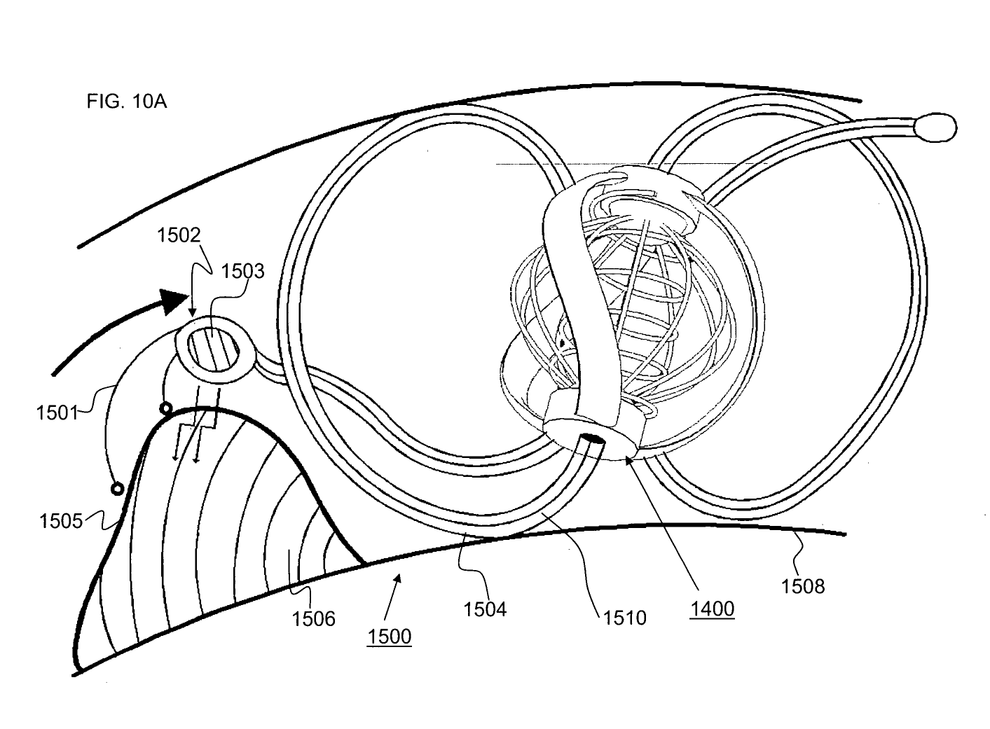

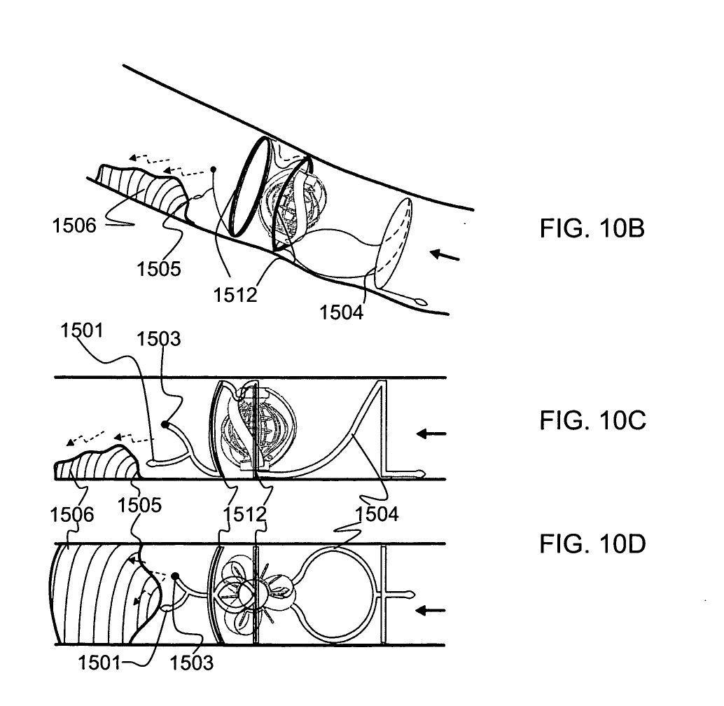

Rotating portion 1400 may also optionally be further attached to a secondary device (described in FIG. 10) converting its mechanical rotational energy to produce an electric charge, optionally to be used to in the treatment of a vulnerable plaque (also described in FIG. 10). Optionally the rotational energy may be used to induce mechanical force to stabilize a vulnerable plaque.

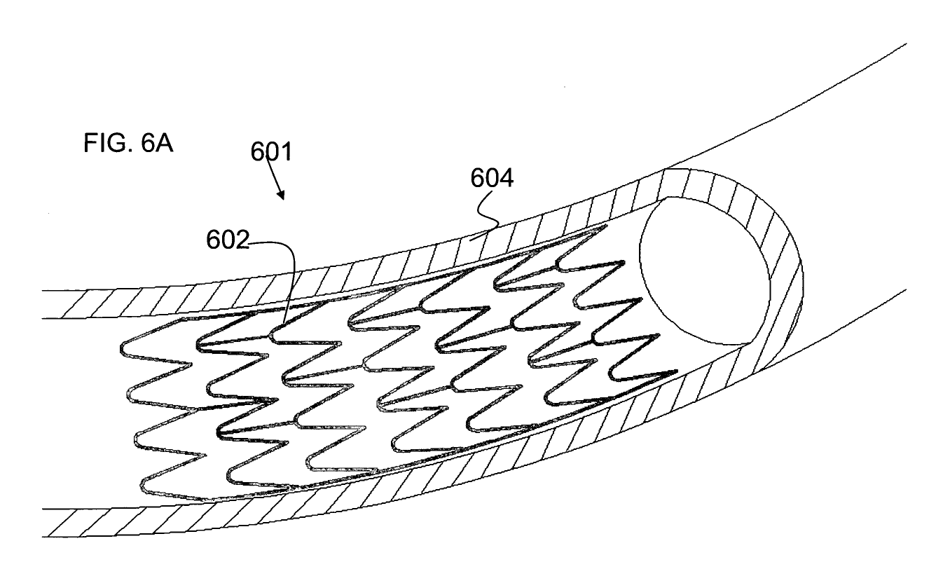

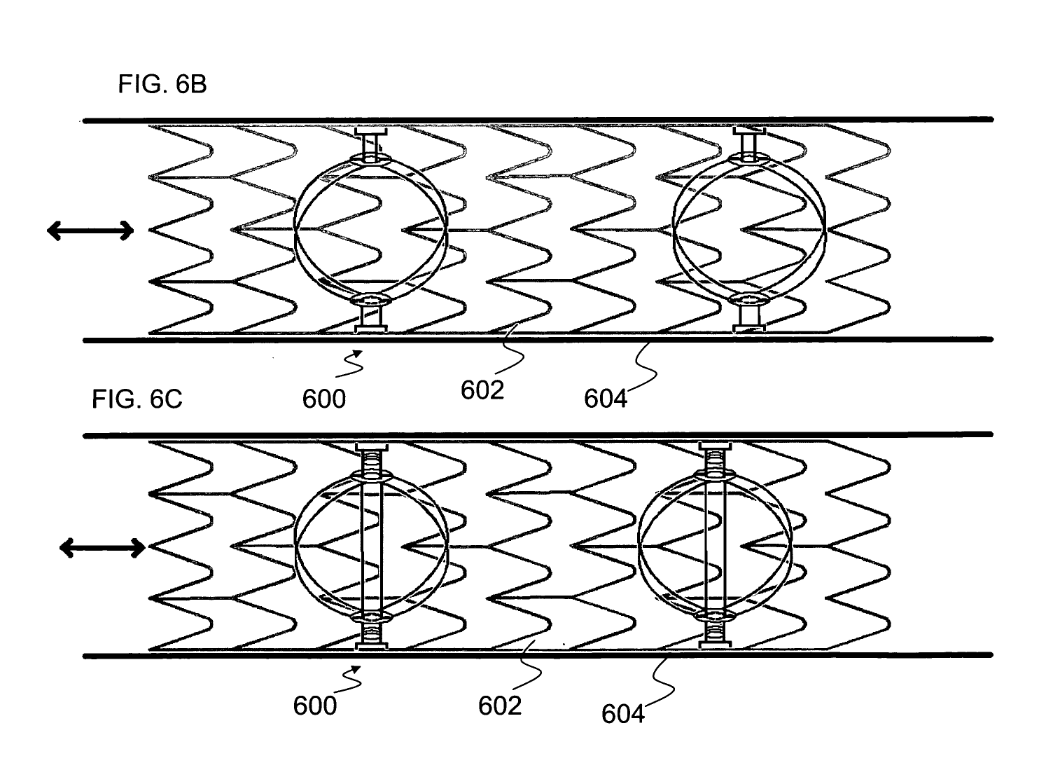

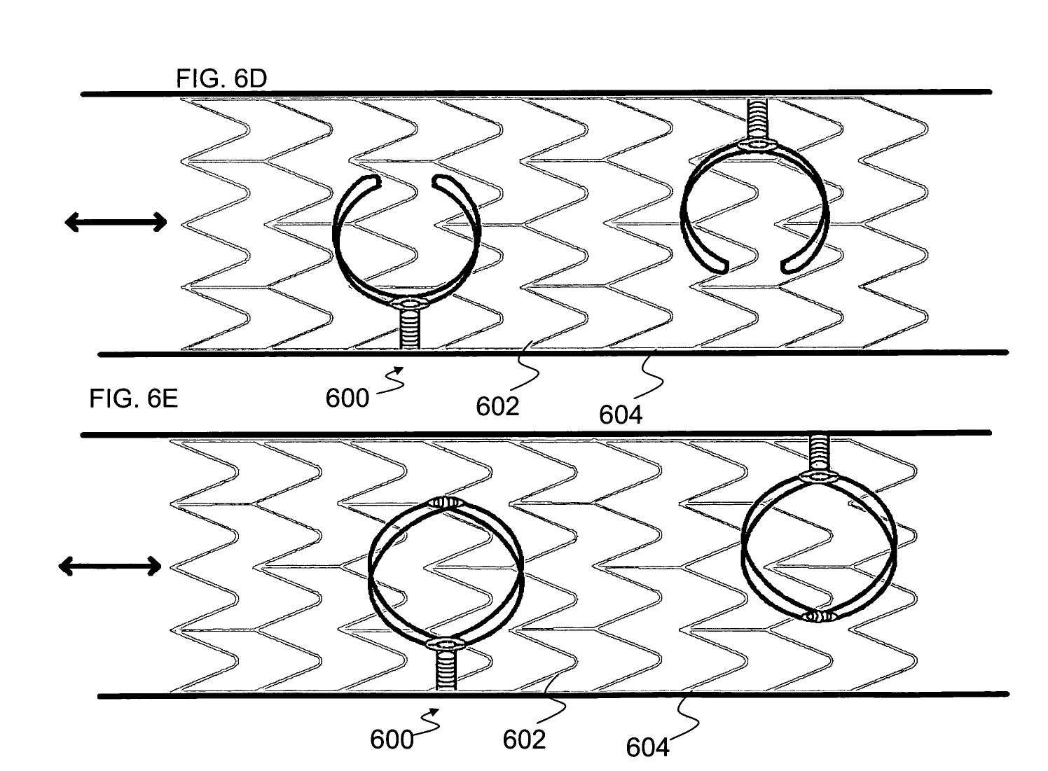

FIG. 6A is a cross-sectional view of a vessel supporting structure 602, which may optionally be implemented in any suitable form, preferably including but not limited to a tubular structure, a stent, blood clot filter, wire frame or stent-graft, imbedded in a blood vessel 601 having blood vessel walls 604. The vessel supporting structure 602 may optionally be fixed with a rotating portion structure (not shown). A rotating portion (not shown) according to the present invention preferably maintains both the structural integrity of blood vessel 601 while maintaining an open lumen allowing blood to flow freely through vessel support structure 602 as the rotating portion breaks down any plaques or other material that may form within or passing through vessel supporting structure 602.

FIG. 6B-G show an optional and exemplary implementation of the present invention, preferably comprising at least one rotating portion 600 according to any one of the rotating portion optional embodiments of the present invention as described in FIGS. 1-5 or any combination thereof in any orientation within a vessel supporting structure 602, which may optionally be implemented in any suitable form, including but not limited to tubular structure, a stent, blood clot filter, wire frame and stent-graft. Installation is within blood vessel wall 604 is preferably achieved by using a stent deployment method as is known in the art and incorporated herein by reference. Blood flows through vessel supporting structure 602 in blood flow direction 601, preferably causing the consecutive rotating portions 600 to spin, optionally and preferably preventing plaque from forming within the lumen of vessel 600, vessel walls 604 and/or vessel support structure 602.

According to a preferred non limiting embodiment of the present invention, a number of rotating portions 600 may be fitted into vessel supporting structure 602 each individually harnessing the hydrokinetic energy of blood flow in order to perform at least one of the rotating portions 600 various uses such as but not limited to maintaining an open lumen, treating vulnerable plaque by applying a mechanical force and/or other energy for example, generating electric charge and so forth.

The number of rotating portions 600 and their orientation in the vessel supporting structure 602 may optionally be varied to meet the various medical application requirements and to enable free blood flow through the vessel supporting structure 602. For example, in order to harvest hydrokinetic energy uniformly and preserve the laminar flow of blood, the rotating portions 600 may optionally be oriented in such way that each rotating portion 600 relates to a different portion of the blood vessel cross section (internal volume). Thus, by preferably not allowing the same blood “packages” or volumes to pass through more than one rotating portion 600, the hydrokinetic energy is harvested more uniformly.

Each rotating portion 600 may optionally be interconnected or function independently of the other. A greater number of rotating portions 600 housed within vessel support structure 602 is preferred as it provides a greater structural integrity.

FIG. 6F shows a system 606 featuring one or more rotating portions 600 that are able not only to spin but also to move vertically along and/or within vessel supporting structure 602.

FIG. 6G shows a system 608 featuring one or more rotating portions 600 that spin freely within the vessel support structure 610 and are enclosed by the vessel support structure 610.

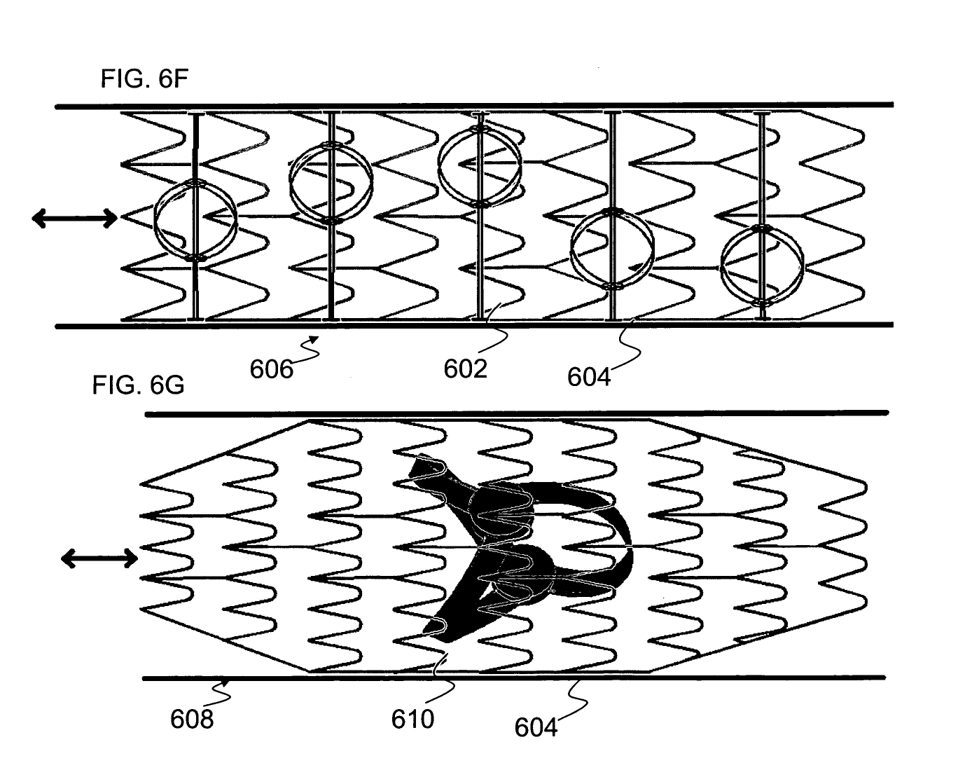

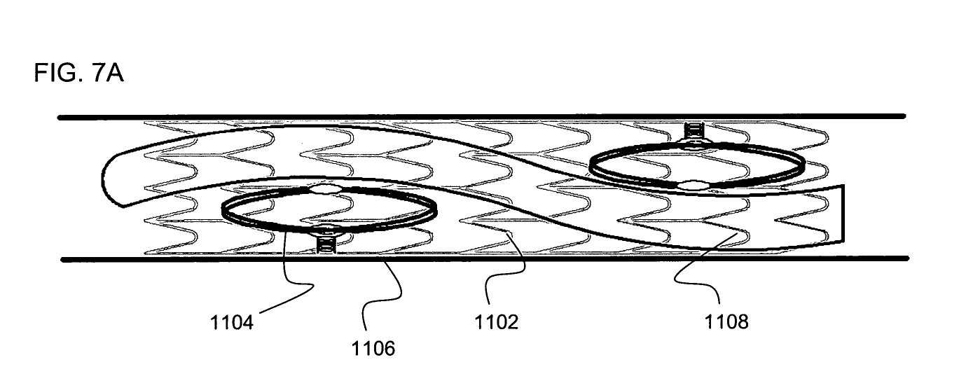

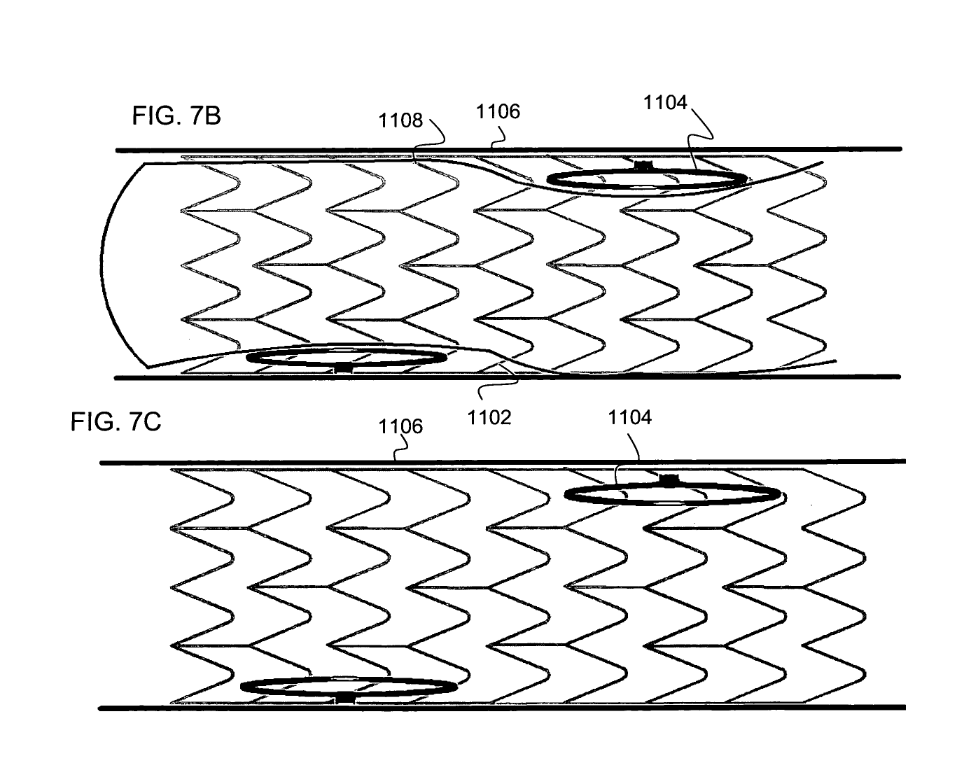

FIG. 7 depicts an optional mode of disabling and condensing the rotating portion of the preferred embodiment of the present invention. Optionally rotating portion 1104 is disabled and condensed by using for example a non-compliant balloon 1108 that may be inflated against the vessel walls 1106 to spread rotating portion 1104 or any other luminal components against the vessel walls 1106. Optionally, the condensed rotating portion and/or filter portion may then be removed from the vasculature. FIG. 7A depicts the non-compliant balloon 1108 entry into the lumen of vessel support structure 1102. FIG. 7B depicts the inflation of the non-compliant balloon 1108 that expands rotating portion 1104 within vessel support structure 1102 spreading it against vessel walls 1106, effectively disabling and expanding it while opening the lumen of vessel structure 1102. FIG. 7C depicts rotating portion 1104 in its expanded state against the vessel wall 1106, after balloon 1108 has been removed.

FIG. 8 depicts another example of how a different form of rotation portion 1104 according to the present invention may be disabled and spread within vessel support structure 1102 by using non compliant balloon 1108. Rotating portion 1104 is preferably disabled by inflating the non compliant balloon 1108, preferably by radial inflation against vessel support structure 1102. FIG. 8A depicts the non-compliant balloon 1108 entry into the lumen of vessel support structure 1102 threaded through rotating portion 1104. FIG. 8B depicts the inflation of balloon 1108 producing radial pressure that spreads rotating portion 1104 against the walls of vessel support structure 1102, effectively disabling and spreading it while opening the lumen of vessel structure 1102. FIG. 8C depicts rotating portion 1104 in its expanded state against vessel wall 1106, after balloon 1108 has been removed.

FIG. 9 depicts an alternative optional method of optionally realigning a shifted or misaligned vessel support structure 1300, comprising a rotating portion 1304 in accordance with any one of the preferred embodiments of the present invention. Vessel support structure 1300 is preferably realigned by using a grasping device 1301, optionally including but not limited to a hook. Grasping device 1301 is preferably associated with a balloon 1308 (for example from an angioplasty catheter), used to preferably realign or optionally remove support structure 1300. By partially inflating balloon 1308 and positioning the semi-inflated balloon 1308 at the center of the blood vessel lumen, the device profile decreases enabling the optional realignment or removal of vessel support structure 1300. Other grasping devices may be used additionally or alternatively.

FIG. 10A depicts an optional, exemplary rotating portion 1400 where a vessel support structure 1504 is coupled thereto, preferably within vessel walls 1508 having vulnerable plaque composed of yellow portion 1506 and white portion 1505. Rotating portion 1400 also preferably features a secondary device 1502, optionally comprising IR and/or UV source 1503, and also preferably electric current source 1501.

Windings 1510 are optionally integrated in vessel support structure 1504 and are preferably able to interact with the magnetically coated blades 1402 to more preferably generate electrical power in windings 1510. Optionally and preferably the produced power is then transferred to secondary device 1502, that may for example optionally be implemented with IR/UV source 1503 for illuminating the vulnerable plaque's white portion 1505. Such illumination optionally heats the external white layer 1505, optionally and preferably stabilizing the vulnerable plaque by hardening white portion 1505.

Optionally and preferably secondary device 1502 may use electric current source 1501 to produce an electric pulse/charge to apply to the white portion 1505, optionally and preferably stabilizing the vulnerable plaque by heating and hardening white portion 1505. Optionally applying heat from different sources including IR, UV or resistive load from secondary device 1502 preferably acts to harden plaque layer 1505. Of course heat and/or electric charge may have other effects, such that the above is provided without wishing to be limited by a single hypothesis.

Preferably the rotating portion 1400 itself may be positioned adjacent to plaque 1506 in order to generate scars to improve healing and to optionally capture and filter the plaque in the event that the plaque bursts. Therefore the position of rotating portion 1400 may optionally serve as a safety factor.

FIGS. 10B-D show an additional, optional implementation of the preferred embodiment as depicted in FIG. 10A where vessel support structure 1504 is formed from axially-extended wires together with relatively flat rings 1512. Rings 1512 are preferably spaced by axially extending connecting wire portion in order to substantially preclude misalignment of the support relative to the axis of a vessel. FIG. 10B depicts a planar view,

FIG. 10C shows a side view, while FIG. 10D shows a top view. Vessel support structure 1504 is preferably placed within vessel walls 1508 having vulnerable plaque composed of yellow portion 1506 and white portion 1505. Other figure reference numbers are as for FIG. 10A.

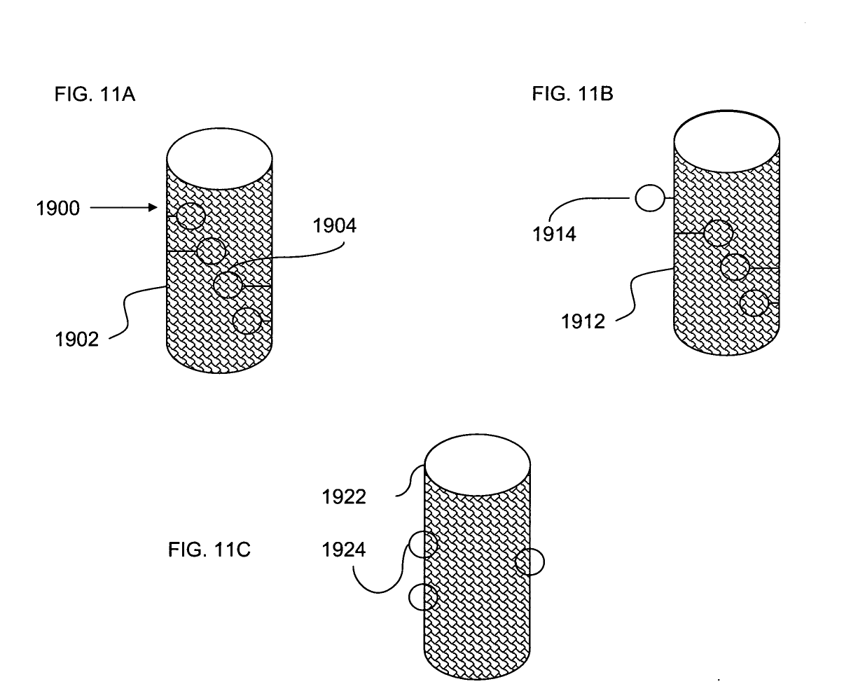

FIG. 11A depicts an exemplary device 1900 according to the present invention comprising vessel support structure 1902 and a plurality of rotating portions 1904 which are preferably placed in a stepwise and sequential manner. Optionally the plurality of rotating portions 1904 spans the inner diameter of vessel support structure 1902. Preferably the use of a vessel support structure 1900 provides an open corridor for blood flow while limiting the level of restenosis to a predefined level. Therefore, even in a situation where vessel support structure 1902 is experiencing restenosis, a passageway or corridor is preferably maintained through which fluids may flow.

FIG. 11B depicts an exemplary embodiment of a vessel support structure 1912, preferably comprising at least one rotating portion 1914, at least one filter portion 1916. Optionally rotating portion 1904 may be placed in a bifurcation junction outside the lumen of vessel support structure 1912 along its outer surface of junction.

FIG. 11C depicts an exemplary embodiment of a vessel support structure 1922, preferably comprising at least one rotating portion 1924 and at least one filter portion 1926. Optionally rotating portion 1924 may be placed along the plane of vessel support structure 1922 integrated within the vessel support structure.

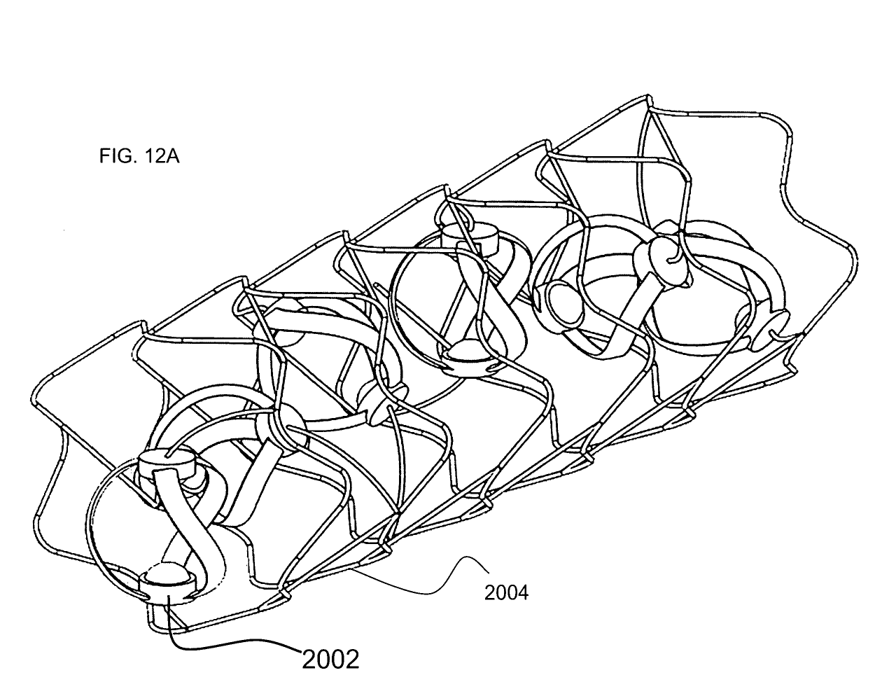

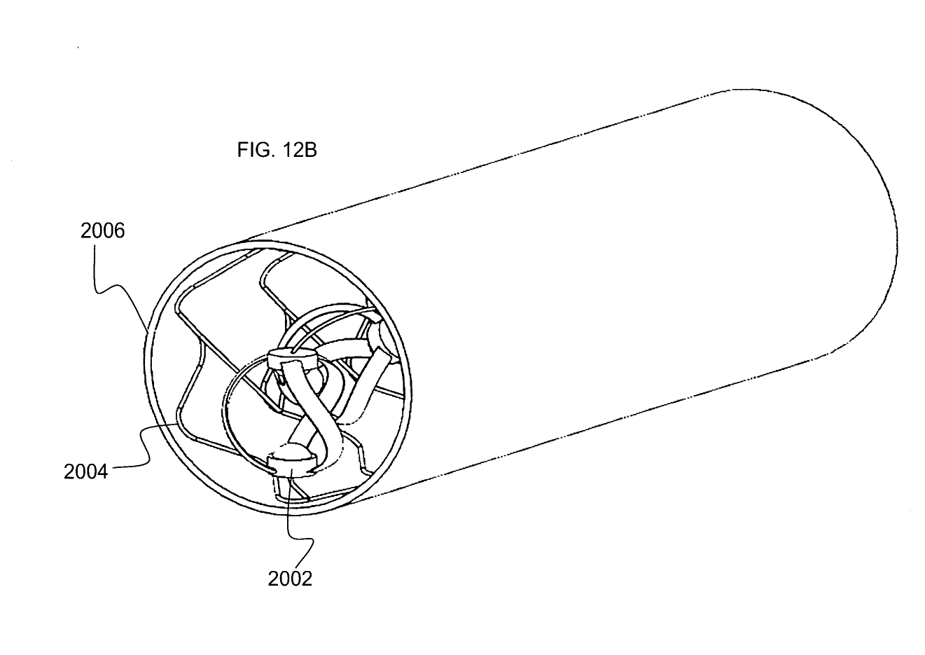

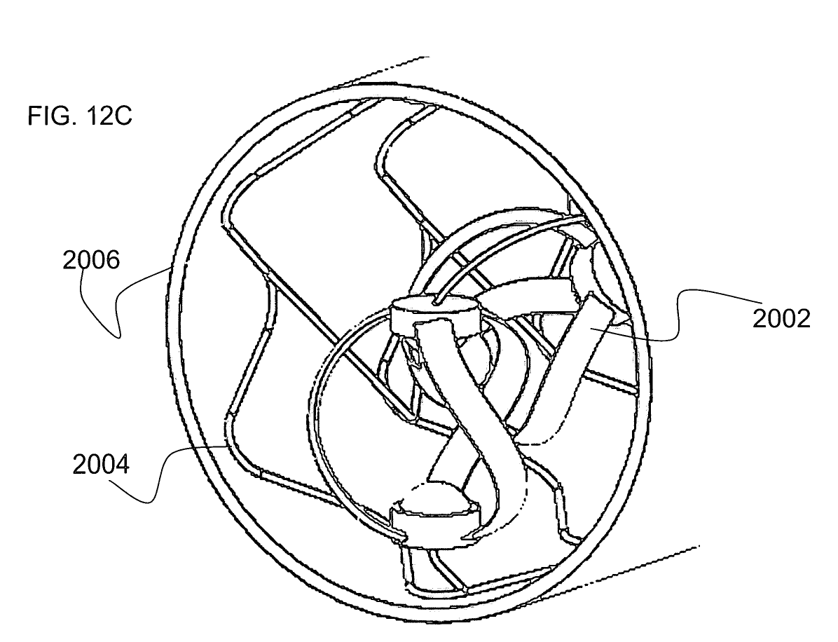

FIG. 12A provides a more detailed depiction of FIG. 11A, showing an optional embodiment according to the present invention wherein a plurality of rotating portions 2002 are preferably placed sequentially and incrementally within a vessel support structure 2004 in a step like manner, to more preferably diagonally span the diameter of the vessel support structure, optionally and preferably providing not only applying mechanical power on a vulnerable plaque but also providing a corridor that limits restenosis levels within the support structure's lumen. FIG. 12B is a depiction of FIG. 12A as implemented within vessel 2006. Similarly, FIG. 12C provides a perspective view of FIG. 12B.

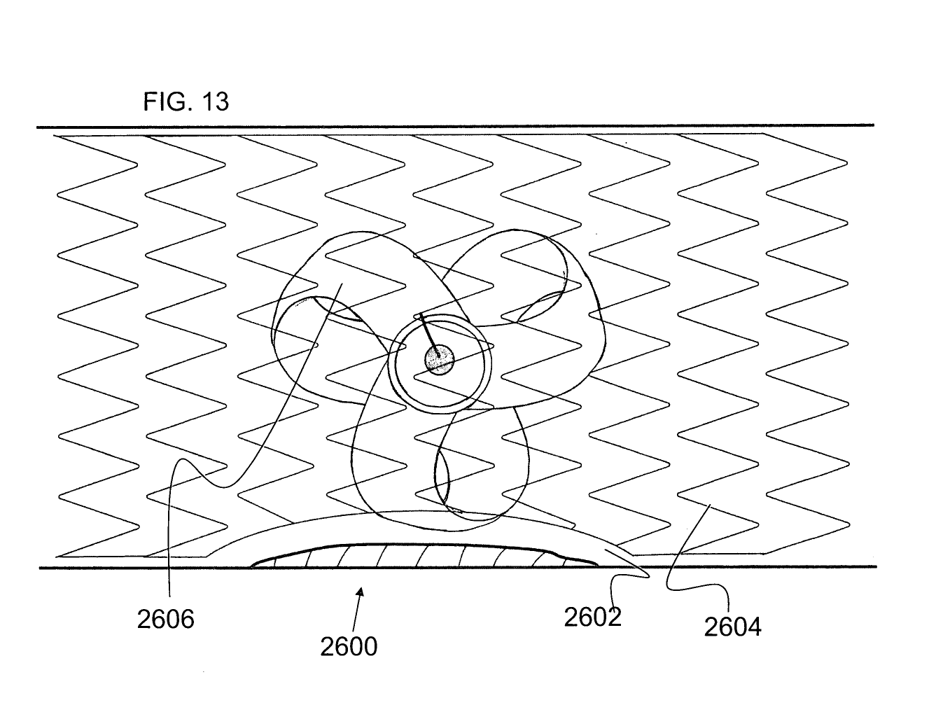

FIG. 13 depicts a device according to an optional embodiment of the present invention for treating a vulnerable plaque 2602. Device 2600 preferably comprises a rotating portion 2606 within a vessel support structure 2604, which is more preferably positioned adjacent to vulnerable plaque 2602; without wishing to be limited by a single hypothesis, such a position may optionally allow the rotating portion 2606 to generate scarring in the outer edge of plaque 2602. Scarring leads to plaque sealing that stabilizes the plaque 2602, reducing a possibility of future fissures or rupture. Preferably device 2600 is able to stabilize vulnerable plaque 2602 and/or prevents the plaque from proliferation or from further development towards rupture.

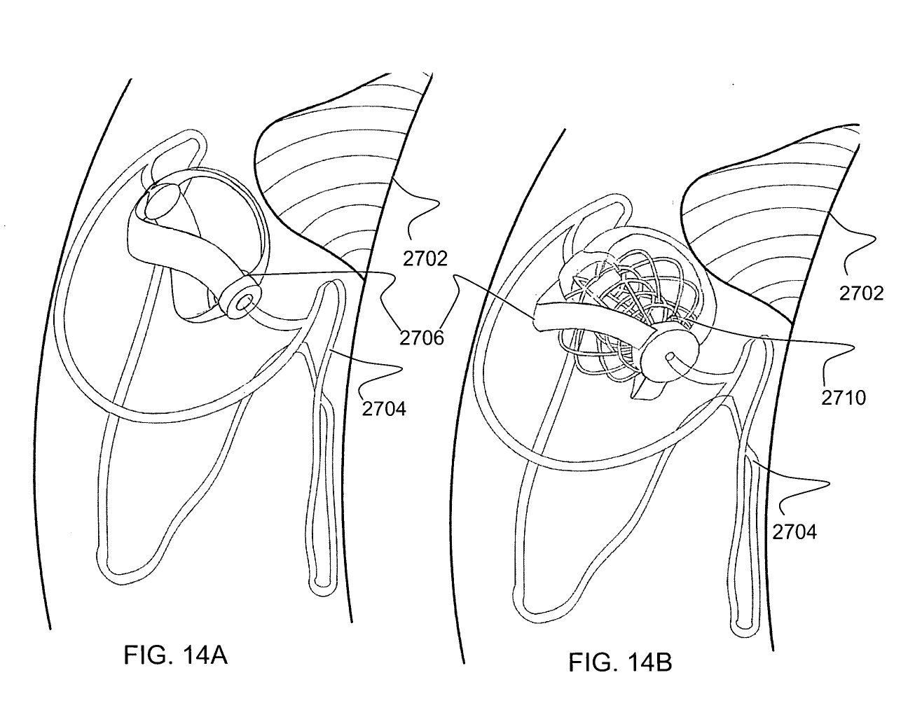

FIG. 14 depicts an additional illustrative embodiment of the device for treating a vulnerable plaque as described in FIG. 13. Device 2700 preferably comprises a rotating portion 2706 and a vessel support structure 2704. Scarring of vulnerable plaque 2702 with rotation portion 2706 preferably leads to its stabilization. Vessel support structure 2704 is optionally implemented as a retrievable tripod or three winged wire frame. Vessel support structure 2704 may optionally take any form for example including a stent. The outer wire frame typically contacts the surrounding blood vessel at some points along the outer wire frame.

Optionally, rotation portion 2706 may be implemented with an inter-blade filter 2710 as depicted in FIG. 14B. Inter-blade filter 2710 optionally catches any debris or the vulnerable plaque 2702 should the vulnerable plaque 2702 erupt while the rotation portion 2706 functions to disrupt it.

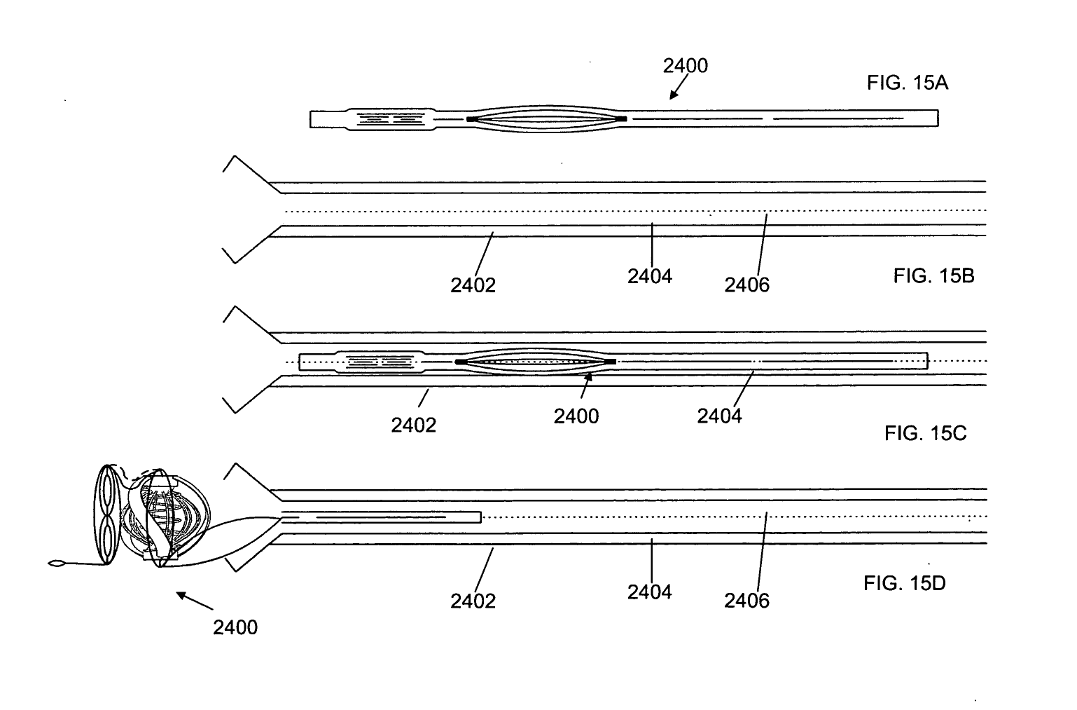

FIGS. 15A-D depict an optional delivery method according to the present invention. FIG. 15A depicts a device 2400 according to an optional embodiment of the present invention, preferably wherein the device 2400 is packaged in its collapsed linear form within a housing catheter in order to assume a delivery state wherein all of the device components are folded onto itself and the platform is straightened to allow delivery through a minimally invasive delivery system. FIG. 15B depicts the catheter, comprising the catheter body 2402, sheath 2404 preferably used to house device 2400 and guidewire 2406 used to deliver and guide device 2400 to the appropriate location. FIG. 15C depicts the loaded catheter ready for delivery and comprising the embodiment of FIG. 15A within the embodiment of FIG. 15B. The delivery catheter and device 2400 are preferably placed within sheath 2404, followed by loading catheter 2402 and deployment to a vessel. Optionally device 2400 may be fabricated in linear form, mainly from a single piece of material so as to not compromise the material properties and strength while reducing the number pieces joined together.

FIG. 15D depicts the delivery of device 2400, as device 2400 is extracted from its housing, it begins to unfold from its linear disposition within the delivery catheter and expand to fill the targeted area. Once device 2400 is delivered, the delivery tools catheter 2402 is preferably removed, leaving the expanded functioning device 2400.

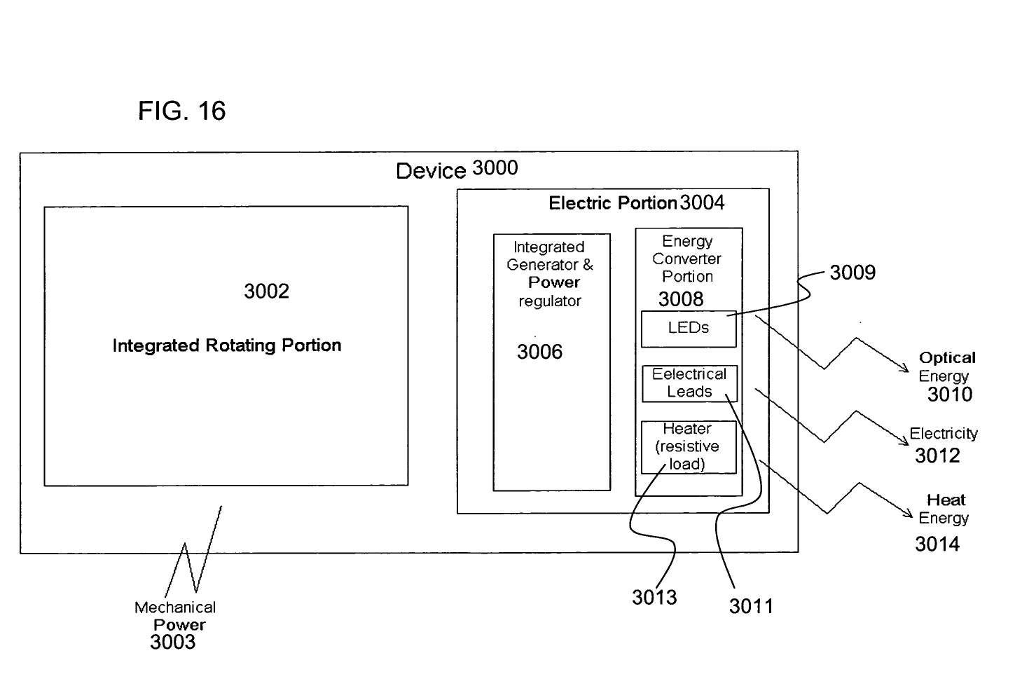

FIG. 16 is a block diagram which depicts in greater detail the exemplary device 1502 described in FIG. 10. Device 3000 comprises integrated rotating portion 3002 and electric portion 3004 that operate together to convert mechanical rotational power 3003 to at least one or more forms of energy. Such a form of energy may optionally include but is not limited to optical energy 3010, electrical energy 3012, or heat 3014. The conversion process is undertaken using electric portion 3004, in which generator 3006 generates and regulates the power produced from the rotational energy. This power is then converted to various forms of energy with energy converter 3004. Optionally energy conversion is able to convert the generated energy to optical energy 3010 using LED 3009, electric energy 3012 using lead 3011, and/or heat energy 3014 using heating leads 3013.

While the invention has been described with respect to a limited number of embodiments, it will be appreciated that many variations, modifications and other applications of the invention may be made.

1. An implantable device for treating a vulnerable plaque, comprising at least one rotating portion comprising a plurality of movable blades and at least one anchor for stably anchoring the rotating portion to a vessel support structure, wherein said vessel support structure is adapted for being implanted near a vulnerable plaque.

2. The implantable device of claim 1 wherein said at least one rotating portion further comprises a filter portion.

3. The implantable device of claim 2 wherein said filter is within a blade rotation volume of said rotating portion.

4.-9. (canceled)

10. The implantable device of claim 1 wherein the vessel support structure takes the form from the group consisting of a tubular structure, stent, a wire frame, a stent graft, ring structure, tripod structure and a winged structure.

11. The implantable device of claim 10 wherein said at least one rotating portion is within the lumen or outside the lumen of the vessel support structure.

12.-17. (canceled)

18. An implantable device for treating a vulnerable plaque, comprising at least one rotating portion comprising a plurality of movable blades, at least one anchor for stably anchoring the rotating portion to said vessel support structure and a peripheral device in communication with said at least one rotating portion, wherein said vessel support structure is adapted for being implanted near a vulnerable plaque.

19. The device of claim 18 wherein the peripheral device is attached to said vessel support structure.

20. The device of claim 18 wherein the peripheral device is attached to said rotating portion.

21. (canceled)

22. The device of claim 18 wherein at least one form of said energy is applied to said vulnerable plaque.

23. (canceled)

24. The device of claim 1 further comprising a dispenser for dispensing a medicament.

25.-28. (canceled)

29. The device of claim 18 further comprising a dispenser for dispensing a medicament.

30-35. (canceled)

36. The device of claim 1 comprising a material selected from the group consisting of metal, plastic, nitinol, titanium, plastically deformable material, temperature-sensitive shape memory material with a transition temperature around body temperature, flexible thread, surgical monofilament sutures, any elastic material and biodegradable material.

37.-38. (canceled)

39. The device of claim 18 comprising a material selected from the group consisting of metal, plastic, nitinol, titanium, plastically deformable material, temperature-sensitive shape memory material with a transition temperature around body temperature, flexible thread, surgical monofilament sutures, any elastic material and biodegradable material.

40. A method for treating a vulnerable plaque within a blood vessel, comprising implanting an implantable device within the blood vessel, said implantable device comprising at least one rotating portion comprising a plurality of movable blades; and rotating said plurality of movable blades to treat the vulnerable plaque.

41. The method of claim 40, wherein said treating comprises inducing scarring in a vulnerable plaque through said rotating of said plurality of movable blades.

42. The method of claim 40, wherein said implantable device further comprises a peripheral device in energetic communication with said at least one rotating portion, wherein said treating comprises providing energy by said peripheral device to the vulnerable plaque.

43. The device of claim 1, provided in a collapsible linear form wherein the device components are folded onto themselves to allow delivery through a minimally invasive delivery system.

44-46. (canceled)

47. A method of determining one or more cardiac parameters, comprising implanting an implantable device within the blood vessel, said implantable device comprising at least one rotating portion comprising a plurality of movable blades; rotating said plurality of movable blades; and determining at least one cardiac parameter according to said rotating.

48. (canceled)

49. The method of claim 47, wherein said at least one cardiovascular parameter comprises one or more of a blood flow rate, cardiac output, vessel occlusion, blood pressure, or temperature.
